SEQUENCE LISTING (219 sequences)

SEQ ID NO: 1 - DNA, 23 bp, Artificial Sequence
  Chemically Synthesized
gacaggacagtgcttgtttaagg

SEQ ID NO: 2 - DNA, 23 bp, Artificial Sequence
  Chemically Synthesized
tgactacacaatattgctcgcac

SEQ ID NO: 3 - DNA, 13139 bp, Mus musculus
  Description Rosa26 locus
aagcttctcacgtagcaaccagagctccagagccagcagctgctgccgccttgtatactcactcctgtgatccaacacaggagcaaccttttctttaccccacccccacttcttaacacacttttttttggggggggggggggaacaagtgctccatgctggaaggattggaactatgcttttagaaaggaacaatcctaaggtcacttttaaattgaggtctttgatttgaaaatcaacaaataccaaattccaaatattcgttttaattaaaccagcaatgtggatataagcattaagttttagttttaaaaaggtcaattttccaaacattcagcaatcatatttaaatttacagctaggaacaagagccttgggtcatgtcctaccaaagaacataactcaatattctacacatgacaatctgaataaccttaaagcctctaatcccataacaggccacaaattttggacagagaactaatgatcctcctgagaaaactggaagaaatccagggaaaagaaattcctgtgtcctccaaactcagaaatctctaattatgtcagtattctctgctttagtcctaggtcagattgcacacatctaaaataacctcttaaagttttcctcctagcgacctaaaccattattaatatcaaattaaccatcaaaacactttcctctcaatatgctgcacacaaacctcctcctggaacctcctccatctggatcctccccaatcaaaagtataggtatttaacatataagcaaggaagtaatgtaaacatgaccttggtcacaaatatgtcatctaaaaacaatttagtcaaggtatggaggaaattcgagaacctgaatctttttaagtattttgagcacaggaacaattggcaaaaggaatccaggtatagacaaaacccagagcccagagctctgggcgaaaaatgagttgctggtgaagacgttacacaagtaacatgagaaagcagaaaatgcaggtcatccacgcacccctgacccaggccagcagggcgggctgcagcatcagtacacaggagaaagatccttattcctaagaatgagaaaggcaaaggcgcccgatagaataaattagcatagaaggggctttcccaggagttaaaactttccttctgagcgattacctactaaaaccagggcttttgcccactaccatttacctaggatcttggcttgcacggattcataggggcatatccctccccctcttctttagagtcgttcttaaaagatcgctctccacgccctaggcagggaaaacgacaaaatctggctcaattccaggctagaaccctacaaattcaacagggatatcgcaaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgagcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatatcccactgaacagcatcaatgggcaaaccccaggttgtttttctgtggagacaaaggtaagatatttcaatatattttcccaagctaatgagatggctcagcaaataatggtactggccattaagtctcatgacctgagcttgatcctcagggaccatgtggtacaaggagagacctaaatccttcagttggacttcaatcttctaccctcatgtccacacacaaataaatacaataaaaaacattctgcagtcgaatttctaaaagggcgaat

SEQ ID NO: 4 - DNA, 1073 bp, Mus musculus
  Description 5′ arm for Rosa26
caggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtct

SEQ ID NO: 5 - DNA, 4333 bp, Mus musculus
  Description 3′ arm for Rosa26
tagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatc

SEQ ID NO: 6 - DNA, 6039 bp, Artificial Sequence
  Chemically Synthesized
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggctagccggccgcgtcgaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggccgacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagcggccgaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttc

SEQ ID NO: 7 - DNA, 14411 bp, Artificial Sequence
  Chemically Synthesized
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggctagccggccgcgtcgaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggccgacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagcggccgaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 8 - DNA, 18866 bp, Artificial Sequence
  Chemically Synthesized
tcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttgatatcgaattctcagaatacaaaacagctttagggactgataaaatatatgtaaaattatgtttgtataataatagaaaaattgagaaataaacataaggctacgtgaaaaggttttgagttcaaaggtatttttctccacgtaatgcacatatacatgtgtgtttatatatgtgcatatgtatatgtagctacatgtgtataaatatattacatatacaagtgtgcacatgtataaacacatacatatgtacacataggtatatatgcatgtatgcattgctagagatgtgattcacatgtctgttgcacatatatacatctgtcaacatacatttcatgtatgcactcttatttcatgagcggtgataagacaccaaaaatgcaccatttacagagagctttgcccttgattggactcactaatgtttgctgaattagaacaaacttctgccttgcccattctgatttaaattagaacaaacttctgccttgcccattctgatttttatcgttagaacaaacttctgccttgcccattctgatttttatcgttagaacaaacttctgccttgcccattctgatttttatcgttagaacaaacttctgccttgcccattctgattttgatttttatcgttgaccattgctctcaggagttaatgtttgatcctggccataaagaaatcgacaatcactgacctatggcctatatttgaggaggaagaagccccttataaaatagccaacagtgggtggcctggcagacagagctgttgtggtcagggggatcaaataggccggcctaggcgcgtaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctcgaccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgcagatccactagttctagggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacttaattaaggccatagcggccatttctagacttagatccccgcgggatcttcaagcagacctacagcaagttcgacacaaactcacacaacgatgacgcactactcaagaactacgggctgctctactgcttcaggaaggacatggacaagg

SEQ ID NO: 9 - DNA, 21730 bp, Artificial Sequence
  Chemically Synthesized
taaagggaacaaaagctgtcgagatctagatatcgatggccatagagttacgctagggataacagggtaatatagccgcggtcaggccctccgagcgtggtggagccgttctgtgagacagccggatcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttcctaggtaaccgatatccctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggctagccggccgcgtcgacttaattaaggccatagcggccattttcgacgacactgcagagacctacttcactaacaaccggtacagttcggtggagtacgcgcccggggagcccaagggcacgccctggcacccgcaccgcggcgaataacttcgtatagcatacattatacgaagttataagctcgatgaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttcagctgctcgactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccctacgcccccaactgagagaactcaaaggttaccccagttggggcactacggatccataacttcgtatagcatacattatacgaagttataccgggccaccggcgcgccatgcctaagaagaagaggaaggttgtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccaaatcggccggcctaggcgcgccggtaaccgaagttcctatactttctagagaataggaacttcggaataggaacttcaagcttaagcgctagcactagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcac

SEQ ID NO: 10 - DNA, 10491 bp, Artificial Sequence
  Chemically Synthesized
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacttaattaaggccatagcggccatttaaatcggccggcctaggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 11 - DNA, 15200 bp, Artificial Sequence
  Chemically Synthesized
ttacgctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgacgccaccatggagaagttcctattccgaagttcctattcttcaaatagtataggaacttcgctcgagctcaagcttcgaattctgcagtcgacggtaccgcgggcccgggatccaccggtcgccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgagcggccgaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgtatgcaggtcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgccatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcattaattaaggccatagcggccatttgggctagcttgcatgcctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagtaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggccctagagcctctgctaaccatgttcatgccttcttctttttcctacagctcctgggcaacgtgctggttgttgtgctgtctcatcattttggcaaagaattacccgccgccaccatggctcccaagaagaagaggaaggtgatgagtcaatttgatatattatgtaaaacaccacctaaggtcctggttcgtcagtttgtggaaaggtttgaaagaccttcaggggaaaaaatagcatcatgtgctgctgaactaacctatttatgttggatgattactcataacggaacagcaatcaagagagccacattcatgagctataatactatcataagcaattcgctgagtttcgatattgtcaacaaatcactccagtttaaatacaagacgcaaaaagcaacaattctggaagcctcattaaagaaattaattcctgcttgggaatttacaattattccttacaatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacctcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaacaaacaggaataccaattattaaaagataacttagtcagatcgtacaacaaggctttgaagaaaaatgcgccttatccaatctttgctataaagaatggcccaaaatctcacattggaagacatttgatgacctcatttctgtcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataatgatctagagtcgagttaggccgcgtcgacctcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagag

SEQ ID NO: 12 - DNA, 5070 bp, Artificial Sequence
  Chemically Synthesized
gggtaactaagtaaggatcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccggagaagttcctattccgaagttcctattcttcaaatagtataggaacttcgctcgagggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatggcgcgtatgcattaattaaggccatagcggccatttatcggccggccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccactggcttctttcttcagtttccttagcactgggaaattgaggaaagaagccggttttttctagagactcgagataacttcgtataatgtatgctatacgaagttatggcgcgttgacattgattattgactagaaatcggccggcctaggcgcgccggtaaccgaagttcctatactttctagagaataggaacttcggaataggaacttcaagccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgg

SEQ ID NO: 13 - DNA, 47 bp, Artificial Sequence
  Chemically Synthesized
tgagaagtctcccagtcagttcaagagactgactgggagacttctca

SEQ ID NO: 14 - DNA, 47 bp, Artificial Sequence
  Chemically Synthesized
gactccagtggtaatctacttcaagagagtagattaccactggagtc

SEQ ID NO: 15 - DNA, 47 bp, Artificial Sequence
  Chemically Synthesized
cggcaggactccgggccgattcaagagatcggcccggagtcctgccg

SEQ ID NO: 16 - DNA, 47 bp, Artificial Sequence
  Chemically Synthesized
cctcatgcagttctctttgttcaagagacaaagagaactgcatgagg

SEQ ID NO: 17 - DNA, 50 bp, Artificial Sequence
  Chemically Synthesized
aagatgaagccactccctatttcaagagaaaatagggagtggcttcatct

SEQ ID NO: 18 - DNA, 41 bp, Artificial Sequence
  Chemically Synthesized
gacagagccaagtggactcacagagtccacttggctctgtc

SEQ ID NO: 19 - DNA, 42 bp, Artificial Sequence
  Chemically Synthesized
ctggacttccagaagaacattcgtgttcttctggaagtccag

SEQ ID NO: 20 - DNA, 47 bp, Artificial Sequence
  Chemically Synthesized
gagattggtccagaacagtttcaagagaactgttctggaccaatctc

SEQ ID NO: 21 - DNA, 47 bp, Artificial Sequence
  Chemically Synthesized
gcccttccgatcatggtagttcaagagactaccatgatcggaagggc

SEQ ID NO: 22 - DNA, 47 bp, Artificial Sequence
  Chemically Synthesized
tctttagaattcttaagtattcaagagatacttaagaattctaaaga

SEQ ID NO: 23 - DNA, 47 bp, Artificial Sequence
  Chemically Synthesized
cattagctatatcaacatgttcaagagacatgttgatatagctaatg

SEQ ID NO: 24 - DNA, 47 bp, Artificial Sequence
  Chemically Synthesized
accacaaacggcggaacgattcaagagatcgttccgccgtttgtggt

SEQ ID NO: 25 - DNA, 47 bp, Artificial Sequence
  Chemically Synthesized
gagggtcttggaggtcttcttcaagagagaagacctccaagaccctc

SEQ ID NO: 26 - DNA, 47 bp, Artificial Sequence
  Chemically Synthesized
gtccatgcccagccgtacattcaagagatgtacggctgggcatggac

SEQ ID NO: 27 - DNA, 47 bp, Artificial Sequence
  Chemically Synthesized
gctggacaccctcgtggagttcaagagactccacgagggtgtccagc

SEQ ID NO: 28 - DNA, 47 bp, Artificial Sequence
  Chemically Synthesized
gaatatcagagaattgagtttcaagagaactcaattctctgatattc

SEQ ID NO: 29 - DNA, 47 bp, Artificial Sequence
  Chemically Synthesized
tggacttcatgaggaaatgttcaagagacatttcctcatgaagtcca

SEQ ID NO: 30 - DNA, 47 bp, Artificial Sequence
  Chemically Synthesized
tattgaatatcctgtggacttcaagagagtccacaggatattcaata

SEQ ID NO: 31 - DNA, 47 bp, Artificial Sequence
  Chemically Synthesized
ttgtactgagagaaactgcttcaagagagcagtttctctcagtacaa

SEQ ID NO: 32 - DNA, 47 bp, Artificial Sequence
  Chemically Synthesized
gatcaatgataggtttgaattcaagagattcaaacctatcattgatc

SEQ ID NO: 33 - DNA, 47 bp, Artificial Sequence
  Chemically Synthesized
ggagtttgagaagtttaaattcaagagatttaaacttctcaaactcc

SEQ ID NO: 34 - DNA, 47 bp, Artificial Sequence
  Chemically Synthesized
gaactcctcgcttgctgagttcaagagactcagcaagcgaggagttc

SEQ ID NO: 35 - DNA, 47 bp, Artificial Sequence
  Chemically Synthesized
ccgaatttaacagagagaattcaagagattctctctgttaaattcgg

SEQ ID NO: 36 - DNA, 47 bp, Artificial Sequence
  Chemically Synthesized
gacagcagaagaatgcagattcaagagatctgcattcttctgctgtc

SEQ ID NO: 37 - DNA, 47 bp, Artificial Sequence
  Chemically Synthesized
ataaagctcaacgagaaccttcaagagaggttctcgttgagctttat

SEQ ID NO: 38 - DNA, 47 bp, Artificial Sequence
  Chemically Synthesized
ggtgaagtggcagaagaatttcaagagaattcttctgccacttcacc

SEQ ID NO: 39 - DNA, 47 bp, Artificial Sequence
  Chemically Synthesized
gtattgcagtaatcatcacttcaagagagtgatgattactgcaatac

SEQ ID NO: 40 - DNA, 47 bp, Artificial Sequence
  Chemically Synthesized
gatatggggttccatgtcattcaagagatgacatggaaccccatatc

SEQ ID NO: 41 - DNA, 47 bp, Artificial Sequence
  Chemically Synthesized
ggagacatggttcttagtgttcaagagacactaagaaccatgtctcc

SEQ ID NO: 42 - DNA, 47 bp, Artificial Sequence
  Chemically Synthesized
agcaccaagttcgtctcagttcaagagactgagacgaacttggtgct

SEQ ID NO: 43 - DNA, 47 bp, Artificial Sequence
  Chemically Synthesized
gatgcaacactgaaagaacttcaagagagttctttcagtgttgcatc

SEQ ID NO: 44 - DNA, 47 bp, Artificial Sequence
  Chemically Synthesized
gtcaatggcagtgatgatattcaagagatatcatcactgccattgac

SEQ ID NO: 45 - DNA, 47 bp, Artificial Sequence
  Chemically Synthesized
cctgctagctgcctgtggcttcaagagagccacaggcagctagcagg

SEQ ID NO: 46 - DNA, 47 bp, Artificial Sequence
  Chemically Synthesized
ccacctttgccagaaggagttcaagagactccttctggcaaaggtgg

SEQ ID NO: 47 - DNA, 47 bp, Artificial Sequence
  Chemically Synthesized
ccctattgaggcaagtgtcttcaagagagacacttgcctcaataggg

SEQ ID NO: 48 - DNA, 47 bp, Artificial Sequence
  Chemically Synthesized FLJ12552/FLJ14256
gaaggaaaacttgctgacgttcaagagacgtcagcaagttttccttc

SEQ ID NO: 49 - DNA, 47 bp, Artificial Sequence
  Chemically Synthesized
ctcacctgggtccatgagattcaagagatctcatggacccaggtgag

SEQ ID NO: 50 - DNA, 47 bp, Artificial Sequence
  Chemically Synthesized
gctgtcttaccgtgtggtcttcaagagagaccacacggtaagacagc

SEQ ID NO: 51 - DNA, 47 bp, Artificial Sequence
  Chemically Synthesized
cctggaccgcatgtatgacttcaagagagtcatacatgcggtccagg

SEQ ID NO: 52 - DNA, 47 bp, Artificial Sequence; identical to SEQ ID NO: 44
  Chemically Synthesized

SEQ ID NO: 53 - DNA, 47 bp, Artificial Sequence; identical to SEQ ID NO: 45
  Chemically Synthesized

SEQ ID NO: 54 - DNA, 47 bp, Artificial Sequence; identical to SEQ ID NO: 46
  Chemically Synthesized

SEQ ID NO: 55 - DNA, 47 bp, Artificial Sequence; identical to SEQ ID NO: 47
  Chemically Synthesized

SEQ ID NO: 56 - DNA, 47 bp, Artificial Sequence
  Chemically Synthesized
ggaaatccgaattgcttggttcaagagaccaagcaattcggatttcc

SEQ ID NO: 57 - DNA, 47 bp, Artificial Sequence
  Chemically Synthesized
cacatttcttcaagtgtggttcaagagaccacacttgaagaaatgtg

SEQ ID NO: 58 - DNA, 47 bp, Artificial Sequence
  Chemically Synthesized
cagcaggatgctcaagaatttcaagagaattcttgagcatcctgctg

SEQ ID NO: 59 - DNA, 47 bp, Artificial Sequence
  Chemically Synthesized
gctgaatacctacattggcttcaagagagccaatgtaggtattcagc

SEQ ID NO: 60 - DNA, 47 bp, Artificial Sequence
  Chemically Synthesized
gggcttgtgcctggccttgttcaagagacaaggccaggcacaagccc

SEQ ID NO: 61 - DNA, 47 bp, Artificial Sequence
  Chemically Synthesized
gccttgtcctgccaagaagttcaagagacttcttggcaggacaaggc

SEQ ID NO: 62 - DNA, 47 bp, Artificial Sequence
  Chemically Synthesized
gattgaagccaagggaacgttcaagagacgttcccttggcttcaatc

SEQ ID NO: 63 - DNA, 47 bp, Artificial Sequence
  Chemically Synthesized
tggcgcctgctccccatctttcaagagaagatggggagcaggcgcca

SEQ ID NO: 64 - DNA, 47 bp, Artificial Sequence
  Chemically Synthesized
gaaccagcaggctctgtggttcaagagaccacagagcctgctggttc

SEQ ID NO: 65 - DNA, 47 bp, Artificial Sequence
  Chemically Synthesized
ggaagcataattatctgccttcaagagaggcagataattatgcttcc

SEQ ID NO: 66 - DNA, 47 bp, Artificial Sequence
  Chemically Synthesized
agaagaagatgcttttcacttcaagagagtgaaaagcatcttcttct

SEQ ID NO: 67 - DNA, 47 bp, Artificial Sequence
  Chemically Synthesized
cttgcagaggaggaacccattcaagagatgggttcctcctctgcaag

SEQ ID NO: 68 - DNA, 47 bp, Artificial Sequence
  Chemically Synthesized
gcaaacaatcagcaatgccttcaagagaggcattgctgattgtttgc

SEQ ID NO: 69 - DNA, 47 bp, Artificial Sequence
  Chemically Synthesized
ttggactgattcatgctatttcaagagaatagcatgaatcagtccaa

SEQ ID NO: 70 - DNA, 47 bp, Artificial Sequence
  Chemically Synthesized
ctggcaattcgttgatgtattcaagagatacatcaacgaattgccag

SEQ ID NO: 71 - DNA, 47 bp, Artificial Sequence
  Chemically Synthesized
ttagatgggcggaagccatttcaagagaatggcttccgcccatctaa

SEQ ID NO: 72 - DNA, 47 bp, Artificial Sequence
  Chemically Synthesized
gaggagtctctgggctcggttcaagagaccgagcccagagactcctc

SEQ ID NO: 73 - DNA, 47 bp, Artificial Sequence
  Chemically Synthesized
gagctgaagggacaagaagttcaagagacttcttgtcccttcagctc

SEQ ID NO: 74 - DNA, 47 bp, Artificial Sequence
  Chemically Synthesized
tgtcgggtagatgacaaggttcaagagaccttgtcatctacccgaca

SEQ ID NO: 75 - DNA, 47 bp, Artificial Sequence
  Chemically Synthesized
cacagctgttcttctgttcttcaagagagaacagaagaacagctgtg

SEQ ID NO: 76 - DNA, 47 bp, Artificial Sequence
  Chemically Synthesized genes
gtggaagcctttacagatcttcaagagagatctgtaaaggcttccac

SEQ ID NO: 77 - DNA, 47 bp, Artificial Sequence
  Chemically Synthesized
caacagctgccttcatctgttcaagagacagatgaaggcagctgttg

SEQ ID NO: 78 - DNA, 47 bp, Artificial Sequence
  Chemically Synthesized
ccataggcagtcctcctaattcaagagattaggaggactgcctatgg

SEQ ID NO: 79 - DNA, 47 bp, Artificial Sequence
  Chemically Synthesized
tgtatcactgccactggttttcaagagaaaccagtggcagtgataca

SEQ ID NO: 80 - DNA, 47 bp, Artificial Sequence
  Chemically Synthesized
catgttgggcagctgcagcttcaagagagctgcagctgcccaacatg

SEQ ID NO: 81 - DNA, 47 bp, Artificial Sequence
  Chemically Synthesized
cacaactggagacctgaagttcaagagacttcaggtctccagttgtg

SEQ ID NO: 82 - DNA, 47 bp, Artificial Sequence
  Chemically Synthesized
gtatgcctccaagaaagagttcaagagactctttcttggaggcatac

SEQ ID NO: 83 - DNA, 47 bp, Artificial Sequence
  Chemically Synthesized
cttcacagtacatttctctttcaagagaagagaaatgtactgtgaag

SEQ ID NO: 84 - DNA, 47 bp, Artificial Sequence
  Chemically Synthesized
gtactttcaaggccggggtttcaagagaaccccggccttgaaagtac

SEQ ID NO: 85 - DNA, 47 bp, Artificial Sequence
  Chemically Synthesized
cttggacaagcaagccaaattcaagagatttggcttgcttgtccaag

SEQ ID NO: 86 - DNA, 47 bp, Artificial Sequence
  Chemically Synthesized
gactattgtgactgatgttttcaagagaaacatcagtcacaatagtc

SEQ ID NO: 87 - DNA, 47 bp, Artificial Sequence
  Chemically Synthesized
ggagaactttctgaagcgcttcaagagagcgcttcagaaagttctcc

SEQ ID NO: 88 - DNA, 47 bp, Artificial Sequence
  Chemically Synthesized
gacgagagaaaccttcaccttcaagagaggtgaaggtttctctcgtc

SEQ ID NO: 89 - DNA, 47 bp, Artificial Sequence
  Chemically Synthesized
acattattctacattctttttcaagagaaaagaatgtagaataatgt

SEQ ID NO: 90 - DNA, 47 bp, Artificial Sequence
  Chemically Synthesized
agattcgcaaatggatgtattcaagagatacatccatttgcgaatct

SEQ ID NO: 91 - DNA, 47 bp, Artificial Sequence
  Chemically Synthesized
cattcccaccatgagtctgttcaagagacagactcatggtgggaatg

SEQ ID NO: 92 - DNA, 47 bp, Artificial Sequence
  Chemically Synthesized
gatcgcccgacacttccgcttcaagagagcggaagtgtcgggcgatc

SEQ ID NO: 93 - DNA, 47 bp, Artificial Sequence
  Chemically Synthesized
ccagcaggcctacgtgctgttcaagagacagcacgtaggcctgctgg

SEQ ID NO: 94 - DNA, 47 bp, Artificial Sequence
  Chemically Synthesized
gccagctcctccacagcacttcaagagagtgctgtggaggagctggc

SEQ ID NO: 95 - DNA, 47 bp, Artificial Sequence
  Chemically Synthesized
cgccgccaagtggagcagattcaagagatctgctccacttggcggcg

SEQ ID NO: 96 - DNA, 47 bp, Artificial Sequence
  Chemically Synthesized
gaagatgcccatgaattccttcaagagaggaattcatgggcatcttc

SEQ ID NO: 97 - DNA, 47 bp, Artificial Sequence
  Chemically Synthesized
caaacaggctgcgccaggcttcaagagagcctggcgcagcctgtttg

SEQ ID NO: 98 - DNA, 47 bp, Artificial Sequence
  Chemically Synthesized
acggcctagcgcctgatggttcaagagaccatcaggcgctaggccgt

SEQ ID NO: 99 - DNA, 47 bp, Artificial Sequence
  Chemically Synthesized
ctgtaacctctctgatcggttcaagagaccgatcagagaggttacag

SEQ ID NO: 100 - DNA, 47 bp, Artificial Sequence
  Chemically Synthesized
tctgtcagtccatcctggcttcaagagagccaggatggactgacaga

SEQ ID NO: 101 - DNA, 47 bp, Artificial Sequence
  Chemically Synthesized
tgaagcgagagtcttgtgattcaagagatcacaagactctcgcttca

SEQ ID NO: 102 - DNA, 47 bp, Artificial Sequence
  Chemically Synthesized
gatggagtgctaatggaaattcaagagatttccattagcactccatc

SEQ ID NO: 103 - DNA, 47 bp, Artificial Sequence
  Chemically Synthesized
ccttcagagattgacacgcttcaagagagcgtgtcaatctctgaagg

SEQ ID NO: 104 - DNA, 47 bp, Artificial Sequence
  Chemically Synthesized
cctgaccacgttccgactgttcaagagacagtcggaacgtggtcagg

SEQ ID NO: 105 - DNA, 47 bp, Artificial Sequence
  Chemically Synthesized
gagttccttcgctgcctgattcaagagatcaggcagcgaaggaactc

SEQ ID NO: 106 - DNA, 47 bp, Artificial Sequence
  Chemically Synthesized
gactgccttgctgccttctttcaagagaagaaggcagcaaggcagtc

SEQ ID NO: 107 - DNA, 47 bp, Artificial Sequence
  Chemically Synthesized
cgccgagggctacgtactcttcaagagagagtacgtagccctcggcg

SEQ ID NO: 108 - DNA, 47 bp, Artificial Sequence
  Chemically Synthesized
ggcgagaagaaaggactgtttcaagagaacagtcctttcttctcgcc

SEQ ID NO: 109 - DNA, 47 bp, Artificial Sequence
  Chemically Synthesized
ggacgagaattgataaagattcaagagatctttatcaattctcgtcc

SEQ ID NO: 110 - DNA, 47 bp, Artificial Sequence
  Chemically Synthesized
gcacgagaatttgggaatcttcaagagagattcccaaattctcgtgc

SEQ ID NO: 111 - DNA, 47 bp, Artificial Sequence
  Chemically Synthesized
ctacttcatgaaatattggttcaagagaccaatatttcatgaagtag

SEQ ID NO: 112 - DNA, 47 bp, Artificial Sequence
  Chemically Synthesized
gataacagcttcttgtctattcaagagatagacaagaagctgttatc

SEQ ID NO: 113 - DNA, 47 bp, Artificial Sequence
  Chemically Synthesized
gagaataggacatcagggcttcaagagagccctgatgtcctattctc

SEQ ID NO: 114 - DNA, 47 bp, Artificial Sequence
  Chemically Synthesized
cttggaagactgaacctgtttcaagagaacaggttcagtcttccaag

SEQ ID NO: 115 - DNA, 47 bp, Artificial Sequence
  Chemically Synthesized
caactcctttgtggatgcattcaagagatgcatccacaaaggagttg

SEQ ID NO: 116 - DNA, 47 bp, Artificial Sequence
  Chemically Synthesized
gatgttgtctccaaatgcattcaagagatgcatttggagacaacatc

SEQ ID NO: 117 - DNA, 47 bp, Artificial Sequence
  Chemically Synthesized
cgtggggactgtacctcccttcaagagagggaggtacagtccccacg

SEQ ID NO: 118 - DNA, 47 bp, Artificial Sequence
  Chemically Synthesized
gtacagcttcagaaccaagttcaagagacttggttctgaagctgtac

SEQ ID NO: 119 - DNA, 47 bp, Artificial Sequence
  Chemically Synthesized
gatgatcttcagagagcaattcaagagattgctctctgaagatcatc

SEQ ID NO: 120 - DNA, 47 bp, Artificial Sequence
  Chemically Synthesized
ggaacatcggaatttgcctttcaagagaaggcaaattccgatgttcc

SEQ ID NO: 121 - DNA, 47 bp, Artificial Sequence
  Chemically Synthesized
gagctagtgagggactcttttcaagagaaagagtccctcactagctc

SEQ ID NO: 122 - DNA, 47 bp, Artificial Sequence
  Chemically Synthesized
gcagggttctttaaggcaattcaagagattgccttaaagaaccctgc

SEQ ID NO: 123 - DNA, 47 bp, Artificial Sequence
  Chemically Synthesized
tcgatgattcctctgaaacttcaagagagtttcagaggaatcatcga

SEQ ID NO: 124 - DNA, 47 bp, Artificial Sequence
  Chemically Synthesized
gataatggaaatattgaacttcaagagagttcaatatttccattatc

SEQ ID NO: 125 - DNA, 47 bp, Artificial Sequence
  Chemically Synthesized
gttcttcatttaaatgatattcaagagatatcatttaaatgaagaac

SEQ ID NO: 126 - DNA, 47 bp, Artificial Sequence
  Chemically Synthesized
gttaacaaacacataaagtttcaagagaactttatgtgtttgttaac

SEQ ID NO: 127 - DNA, 47 bp, Artificial Sequence
  Chemically Synthesized
gttagagaagattcttcgtttcaagagaacgaagaatcttctctaac

SEQ ID NO: 128 - DNA, 47 bp, Artificial Sequence
  Chemically Synthesized
gttgattggacaattaaacttcaagagagtttaattgtccaatcaac

SEQ ID NO: 129 - DNA, 47 bp, Artificial Sequence
  Chemically Synthesized
ggttgataccgtaaagcgcttcaagagagcgctttacggtatcaacc

SEQ ID NO: 130 - DNA, 47 bp, Artificial Sequence
  Chemically Synthesized
gcaatgaaacgtccaatggttcaagagaccattggacgtttcattgc

SEQ ID NO: 131 - DNA, 47 bp, Artificial Sequence
  Chemically Synthesized
agctagagaaaattcttcgttcaagagacgaagaattttctctagct

SEQ ID NO: 132 - DNA, 47 bp, Artificial Sequence
  Chemically Synthesized
gatcctatatgatggatgattcaagagatcatccatcatataggatc

SEQ ID NO: 133 - DNA, 47 bp, Artificial Sequence
  Chemically Synthesized
gttcttcttgtcagtgaaattcaagagatttcactgacaagaagaac

SEQ ID NO: 134 - DNA, 47 bp, Artificial Sequence
  Chemically Synthesized
cttgagcttgagtgaccacttcaagagagtggtcactcaagctcaag

SEQ ID NO: 135 - DNA, 47 bp, Artificial Sequence
  Chemically Synthesized
gaccggccagcgagtctacttcaagagagtagactcgctggccggtc

SEQ ID NO: 136 - DNA, 47 bp, Artificial Sequence
  Chemically Synthesized
ggacctgggctacatctacttcaagagagtagatgtagcccaggtcc

SEQ ID NO: 137 - DNA, 47 bp, Artificial Sequence
  Chemically Synthesized
ctctgtggtccaggtgctcttcaagagagagcacctggaccacagag

SEQ ID NO: 138 - DNA, 47 bp, Artificial Sequence
  Chemically Synthesized
gaccacacgatttgcctcattcaagagatgaggcaaatcgtgtggtc

SEQ ID NO: 139 - DNA, 47 bp, Artificial Sequence
  Chemically Synthesized
tggcttgtttattgaaggattcaagagatccttcaataaacaagcca

SEQ ID NO: 140 - DNA, 47 bp, Artificial Sequence
  Chemically Synthesized
gtgaatttggggaagataattcaagagattatcttccccaaattcac

SEQ ID NO: 141 - DNA, 47 bp, Artificial Sequence
  Chemically Synthesized
cgctatagcttgaatgagtttcaagagaactcattcaagctatagcg

SEQ ID NO: 142 - DNA, 47 bp, Artificial Sequence
  Chemically Synthesized
gatatcctggctccacacattcaagagatgtgtggagccaggatatc

SEQ ID NO: 143 - DNA, 47 bp, Artificial Sequence
  Chemically Synthesized
gagccagtcggatgtagatttcaagagaatctacatccgactggctc

SEQ ID NO: 144 - DNA, 47 bp, Artificial Sequence
  Chemically Synthesized
gtaaattctgaaggcgaatttcaagagaattcgccttcagaatttac

SEQ ID NO: 145 - DNA, 47 bp, Artificial Sequence
  Chemically Synthesized
gccctcctaaatcaggcaattcaagagattgcctgatttaggagggc

SEQ ID NO: 146 - DNA, 47 bp, Artificial Sequence
  Chemically Synthesized
gttgagaaatggagtgaagttcaagagacttcactccatttctcaac

SEQ ID NO: 147 - DNA, 47 bp, Artificial Sequence
  Chemically Synthesized
gcttggaaaatgcaaggcgttcaagagacgccttgcattttccaagc

SEQ ID NO: 148 - DNA, 47 bp, Artificial Sequence
  Chemically Synthesized
ctgcatcatagaccagatcttcaagagagatctggtctatgatgcag

SEQ ID NO: 149 - DNA, 47 bp, Artificial Sequence
  Chemically Synthesized
gatcaccacgtatgtgtccttcaagagaggacacatacgtggtgatc

SEQ ID NO: 150 - DNA, 47 bp, Artificial Sequence
  Chemically Synthesized
tgacaacaagtattccctgttcaagagacagggaatacttgttgtca

SEQ ID NO: 151 - DNA, 47 bp, Artificial Sequence
  Chemically Synthesized
gaaatataagacagattccttcaagagaggaatctgtcttatatttc

SEQ ID NO: 152 - DNA, 47 bp, Artificial Sequence
  Chemically Synthesized
cccatcaagtttagaggatttcaagagaatcctctaaacttgatggg

SEQ ID NO: 153 - DNA, 47 bp, Artificial Sequence
  Chemically Synthesized
ggtgtcccatgggaatatattcaagagatatattcccatgggacacc

SEQ ID NO: 154 - DNA, 47 bp, Artificial Sequence
  Chemically Synthesized
gaatgccgacctacaaagattcaagagatctttgtaggtcggcattc

SEQ ID NO: 155 - DNA, 47 bp, Artificial Sequence
  Chemically Synthesized
cagttatattctgtgatgtttcaagagaacatcacagaatataactg

SEQ ID NO: 156 - DNA, 47 bp, Artificial Sequence
  Chemically Synthesized
gaggtgttggggacaaaggttcaagagacctttgtccccaacacctc

SEQ ID NO: 157 - DNA, 47 bp, Artificial Sequence
  Chemically Synthesized
gtgggctcattggctgaagttcaagagacttcagccaatgagcccac

SEQ ID NO: 158 - DNA, 47 bp, Artificial Sequence
  Chemically Synthesized
gagctactgaggacagaaattcaagagatttctgtcctcagtagctc

SEQ ID NO: 159 - DNA, 47 bp, Artificial Sequence
  Chemically Synthesized
tcagcaggatgctcaggagttcaagagactcctgagcatcctgctga

SEQ ID NO: 160 - DNA, 47 bp, Artificial Sequence
  Chemically Synthesized
gaagttctccatccagaggttcaagagacctctggatggagaacttc

SEQ ID NO: 161 - DNA, 47 bp, Artificial Sequence
  Chemically Synthesized
gccggtccccaccagcagcttcaagagagctgctggtggggaccggc

SEQ ID NO: 162 - DNA, 47 bp, Artificial Sequence
  Chemically Synthesized
cactcgggagttgagagatttcaagagaatctctcaactcccgagtg

SEQ ID NO: 163 - DNA, 47 bp, Artificial Sequence
  Chemically Synthesized
gcccttgggtctgtttgacttcaagagagtcaaacagacccaagggc

SEQ ID NO: 164 - DNA, 47 bp, Artificial Sequence
  Chemically Synthesized
ctcaacactaaacagcaagttcaagagacttgctgtttagtgttgag

SEQ ID NO: 165 - DNA, 47 bp, Artificial Sequence
  Chemically Synthesized
gatttcattggacagcatattcaagagatatgctgtccaatgaaatc

SEQ ID NO: 166 - DNA, 47 bp, Artificial Sequence
  Chemically Synthesized
catggggcaccaactaattttcaagagaaattagttggtgccccatg

SEQ ID NO: 167 - DNA, 47 bp, Artificial Sequence
  Chemically Synthesized
ggtgtctctgcgggattgtttcaagagaacaatcccgcagagacacc

SEQ ID NO: 168 - DNA, 47 bp, Artificial Sequence
  Chemically Synthesized
agttcagtaggtgtagactttcaagagaagtctacacctactgaact

SEQ ID NO: 169 - DNA, 47 bp, Artificial Sequence
  Chemically Synthesized
gagttcctgaagctcctcattcaagagatgaggagcttcaggaactc

SEQ ID NO: 170 - DNA, 47 bp, Artificial Sequence
  Chemically Synthesized
ggatttgctgggggcaaggttcaagagaccttgcccccagcaaatcc

SEQ ID NO: 171 - DNA, 47 bp, Artificial Sequence
  Chemically Synthesized
ctcagaaagccaacattcattcaagagatgaatgttggctttctgag

SEQ ID NO: 172 - DNA, 47 bp, Artificial Sequence
  Chemically Synthesized
cgcattgtaataagaaggtttcaagagaaccttcttattacaatgcg

SEQ ID NO: 173 - DNA, 47 bp, Artificial Sequence
  Chemically Synthesized
gggaggaaaatgcagaaatttcaagagaatttctgcattttcctccc

SEQ ID NO: 174 - DNA, 47 bp, Artificial Sequence
  Chemically Synthesized
ttacaaatttaggaaatacttcaagagagtatttcctaaatttgtaa

SEQ ID NO: 175 - DNA, 47 bp, Artificial Sequence
  Chemically Synthesized
gttatgaattgatatgcagttcaagagactgcatatcaattcataac

SEQ ID NO: 176 - DNA, 47 bp, Artificial Sequence
  Chemically Synthesized
gtgataacacaactaatggttcaagagaccattagttgtgttatcac

SEQ ID NO: 177 - DNA, 47 bp, Artificial Sequence
  Chemically Synthesized
gtagaggagagttctgaaattcaagagatttcagaactctcctctac

SEQ ID NO: 178 - DNA, 47 bp, Artificial Sequence
  Chemically Synthesized
gcctctaatcctgataaggttcaagagaccttatcaggattagaggc

SEQ ID NO: 179 - DNA, 47 bp, Artificial Sequence
  Chemically Synthesized
gatgatcttcaggctgccattcaagagatggcagcctgaagatcatc

SEQ ID NO: 180 - DNA, 47 bp, Artificial Sequence
  Chemically Synthesized
gtatggacaagagcgttggttcaagagaccaacgctcttgtccatac

SEQ ID NO: 181 - DNA, 47 bp, Artificial Sequence
  Chemically Synthesized
cgaacccttctggaacagtttcaagagaactgttccagaagggttcg

SEQ ID NO: 182 - DNA, 47 bp, Artificial Sequence
  Chemically Synthesized
gtggcatgaagattatagtttcaagagaactataatcttcatgccac

SEQ ID NO: 183 - DNA, 47 bp, Artificial Sequence
  Chemically Synthesized
ggtgaacaaggacagtatcttcaagagagatactgtccttgttcacc

SEQ ID NO: 184 - DNA, 47 bp, Artificial Sequence
  Chemically Synthesized
gcaatagaggatgattctgttcaagagacagaatcatcctctattgc

SEQ ID NO: 185 - DNA, 47 bp, Artificial Sequence
  Chemically Synthesized
tctgtgaatgccaaagttcttcaagagagaactttggcattcacaga

SEQ ID NO: 186 - DNA, 47 bp, Artificial Sequence
  Chemically Synthesized
cacaccagggaaggtctagttcaagagactagaccttccctggtgtg

SEQ ID NO: 187 - DNA, 47 bp, Artificial Sequence
  Chemically Synthesized
gcaggaagatgcccatgaattcaagagattcatgggcatcttcctgc

SEQ ID NO: 188 - DNA, 47 bp, Artificial Sequence
  Chemically Synthesized
gaatgtgcaatatcctgagttcaagagactcaggatattgcacattc

SEQ ID NO: 189 - DNA, 47 bp, Artificial Sequence
  Chemically Synthesized
tggatgatgccaaggtcacttcaagagagtgaccttggcatcatcca

SEQ ID NO: 190 - DNA, 47 bp, Artificial Sequence
  Chemically Synthesized
gctccgtgctaaacctctcttcaagagagagaggtttagcacggagc

SEQ ID NO: 191 - DNA, 47 bp, Artificial Sequence
  Chemically Synthesized
gcctccacctcaacagaggttcaagagacctctgttgaggtggaggc

SEQ ID NO: 192 - DNA, 47 bp, Artificial Sequence
  Chemically Synthesized
ctgcatcatagaccaaatcttcaagagagatttggtctatgatgcag

SEQ ID NO: 193 - DNA, 47 bp, Artificial Sequence
  Chemically Synthesized
gatcactacatacatttccttcaagagaggaaatgtatgtagtgatc

SEQ ID NO: 194 - DNA, 47 bp, Artificial Sequence
  Chemically Synthesized
gtaaagagagcagaatgaattcaagagattcattctgctctctttac

SEQ ID NO: 195 - DNA, 47 bp, Artificial Sequence
  Chemically Synthesized
cgcggggcgcagtggtatcttcaagagagataccactgcgccccgcg

SEQ ID NO: 196 - DNA, 47 bp, Artificial Sequence
  Chemically Synthesized
cagaaggcagtggggaagattcaagagatcttccccactgccttctg

SEQ ID NO: 197 - DNA, 47 bp, Artificial Sequence
  Chemically Synthesized
gcctgggagaatcacaggtttcaagagaacctgtgattctcccaggc

SEQ ID NO: 198 - DNA, 47 bp, Artificial Sequence
  Chemically Synthesized
accagacaaggaaatacccttcaagagagggtatttccttgtctggt

SEQ ID NO: 199 - DNA, 47 bp, Artificial Sequence
  Chemically Synthesized
cacatccaccacatcgaccttcaagagaggtcgatgtggtggatgtg

SEQ ID NO: 200 - DNA, 47 bp, Artificial Sequence
  Chemically Synthesized
gtcacaacccaagaccatgttcaagagacatggtcttgggttgtgac

SEQ ID NO: 201 - DNA, 47 bp, Artificial Sequence
  Chemically Synthesized
ctcaacaggacaaatcccattcaagagatgggatttgtcctgttgag

SEQ ID NO: 202 - DNA, 47 bp, Artificial Sequence
  Chemically Synthesized
tagatcaattattgtggatttcaagagaatccacaataattgatcta

SEQ ID NO: 203 - DNA, 47 bp, Artificial Sequence
  Chemically Synthesized
ggaacaccttattgatgaattcaagagattcatcaataaggtgttcc

SEQ ID NO: 204 - DNA, 47 bp, Artificial Sequence
  Chemically Synthesized
ctttaacagaaattgtctcttcaagagagagacaatttctgttaaag

SEQ ID NO: 205 - DNA, 47 bp, Artificial Sequence
  Chemically Synthesized
cctatgcagtacaaagtggttcaagagaccactttgtactgcatagg

SEQ ID NO: 206 - DNA, 47 bp, Artificial Sequence
  Chemically Synthesized
gatcttttcttgctttggattcaagagatccaaagcaagaaaagatc

SEQ ID NO: 207 - DNA, 47 bp, Artificial Sequence
  Chemically Synthesized
cagcatccttcaggccttattcaagagataaggcctgaaggatgctg

SEQ ID NO: 208 - DNA, 47 bp, Artificial Sequence
  Chemically Synthesized
gatagtgactcggatctgcttcaagagagcagatccgagtcactatc

SEQ ID NO: 209 - DNA, 47 bp, Artificial Sequence
  Chemically Synthesized
gacatcacagcccgggagtttcaagagaactcccgggctgtgatgtc

SEQ ID NO: 210 - DNA, 47 bp, Artificial Sequence
  Chemically Synthesized
ggacacagcctatgtgctgttcaagagacagcacataggctgtgtcc

SEQ ID NO: 211 - DNA, 47 bp, Artificial Sequence
  Chemically Synthesized
gtggaggagatctacgaccttcaagagaggtcgtagatctcctccac

SEQ ID NO: 212 - DNA, 47 bp, Artificial Sequence
  Chemically Synthesized
ctcttgtgcaactcatgccttcaagagaggcatgagttgcacaagag

SEQ ID NO: 213 - DNA, 47 bp, Artificial Sequence
  Chemically Synthesized
acagggcccctgcagcctcttcaagagagaggctgcaggggccctgt

SEQ ID NO: 214 - DNA, 47 bp, Artificial Sequence
  Chemically Synthesized
gaagacctggcggcaggtgttcaagagacacctgccgccaggtcttc

SEQ ID NO: 215 - DNA, 21 bp, Artificial Sequence
  Chemically Synthesized
gcctgtgcctcttcagctacc

SEQ ID NO: 216 - DNA, 21 bp, Artificial Sequence
  Chemically Synthesized
gcggagacagcgacgaagagc

SEQ ID NO: 217 - DNA, 19 bp, Artificial Sequence
  Chemically Synthesized
cttattggagagagcacga

SEQ ID NO: 218 - DNA, 9169 bp, Artificial Sequence
  Chemically Synthesized
gggtaactaagtaaggatcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccggagaagttcctattccgaagttcctattcttcaaatagtataggaacttcgctcgagggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatggcgcgtatgcattaattaaggccatagcggccatttaaatcggccggccatttaaatagttacgctagggataacagggtaatatagttaattaatctagaactagtggatccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggatggagccgatattggattcaagagattcaatattggcttcatccactttttggcgcgtgctagcccgggctagcttgcatgcctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggcaattgtttaaactcgaggatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccacgcgccggtaaccgaagttcctatactttctagagaataggaacttcggaataggaacttcaagccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgg

SEQ ID NO: 219 - DNA, 5778 bp, Artificial Sequence
  Chemically Synthesized
cctaggtaaccgatatccctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggctagccggccgcgtcgacttaattaaggccatagcggccattttcgacgacactgcagagacctacttcactaacaaccggtacagttcggtggagtacgcgcccggggagcccaagggcacgccctggcacccgcaccgcggcgaataacttcgtatagcatacattatacgaagttatacggcgcgccatgcctaagaagaagaggaaggttgtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccaaatcggccggcctaggcgcgccggtaaccgaagttcctatactttctagagaataggaacttcggaataggaacttcaagcttaagcgctagcac

(19) United States
(12) Patent Application Publication
(10) Pub. No.: US 2008/0313747 A1
(43) Pub. Date: Dec. 18, 2008

(54) Targeted Transgenesis of Short Hairpin Rna Expression Cassettes Using Recombinase Mediated Cassette Exchange

(76) Inventors: Heidrun Kern, Monheim (DE); Jost Seibler, Koln (DE); Frieder Schwenk, Koln (DE)

Correspondence Address: NORRIS, MCLAUGHLIN & MARCUS, PA, 875 THIRD AVENUE, 18TH FLOOR, NEW YORK, NY, 10022, US

(21) Appl. No.: 11/571,194

(22) PCT Filed: Jul. 7, 2005
(86) PCT No.: PCT/EP2005/053245

§ 371 Date: Feb. 20, 2007

(30) Foreign Application Priority Data
Jul. 7, 2004 (EP) 04103229.3

(51) Int. Cl. A01K 67/027; C12N 15/62; C12N 5/06; C12Q 1/02; C12N 1/19

(52) U.S. Cl. 800/3; 435/455; 435/325; 435/252.3; 800/21; 800/13; 435/29

(57) ABSTRACT

The invention provides a method for targeted transgenesis of short hairpin RNA expression cassettes using recombinase mediated cassette exchange. Suitable nucleotide acid sequences and vectors for the targeted transgenesis and recombinase mediated transgenesis are provided.

### INTRODUCTION

The invention provides a method for targeted transgenesis of short hairpin RNA expression cassettes using recombinase mediated cassette exchange. Suitable nucleotide acid sequences and vectors for the targeted transgenesis and recombinase mediated transgenesis are provided.

### BACKGROUND OF THE INVENTION

The generation of transgenic mice by nuclear injection of purified DNA into fertilized eggs is a widely used approach for studying gene or promoter function in vivo. However, the level and pattern of expression often varies strongly depending on copy number, configuration, and integration site of the transgene. In addition, founder mice occasionally do not transmit the transgene. Thus, a number of different founders need to be generated and tested in order to identify a useful strain, which is a laborious and time-consuming undertaking (Bradley et. al., Nature Genet., 14:121-123 (1996); Jasin et al., Proc. Natl. Acad. Sci. USA, 93:8804-8808 (1996); Dobie et al., Trends Genet., 13:127-130 (1997); Garrick et al., Nature Genet., 18:56-59 (1998), Al-Shawl et al., Mol. Cell. Boil. 10:1192-1198 (1990)).

To overcome these limitations, homologous recombination in embryonic stem cells has been used to produce mice carrying a single copy of the transgene integrated into a predetermined site of the genome (Shaw-White et al., Transgenic Res.; (1):1-13 (1993); Bronson et al., Proc. Natl. Acad. Sci. USA, 93(17:9067-72 (1996); Hatada et al., J. Biol., Chem., 274(2):948-55 (1999); Vivian et al., Biotechniques, 27(1):154-62 (1999); Evans et al., Physiol. Genomics, Mar. 13, 2(2):67-75 (2000); Cvetkovic et al., J. boil. Chem., 275(2):1073-8 (2000); Guillot et al., Physiol. Genomics, Mar. 13, (2):77-83 (2000); Magness et al., Blood, 95(11):3568-77 (2000); Misra et al., BMC Biotechnol., 1(1):12 (2001); Minami et al., Blood, 100(12):4019-25 (2002); Tang et al., Genesis, 32(3):199-202 (2002)). In these studies, the ubiquitous Hprt locus was more or less successfully used for ‘targeted transgenesis’. Insertion of a lacZ gene under the control of the polyoma enhancer/HSV thymidine kinase promoter into the third exon of Hprt resulted in variable B-galactosidase expression that was both orientation and cell-type dependent (Shaw-White et al., Transgenic Res.; (1):1-13 (1993)). Although transgenes under the control of the human and the chicken β-actin gene promoter resulted in widespread expression when inserted into the Hprt locus, the level of transcripts varied strongly in different tissues (Bronson et al., Proc. Natl. Acad. Sci. USA, 93(17:9067-72 (1996)). Unexpectedly, expression of these transgenes, but not of the endogenous Hprt gene appeared to be low or undetectable in kidney and liver (Bronson et al., Proc. Natl. Acad. Sci. USA, 93(17:9067-72 (1996)). Hatada et al. demonstrated that the HPRT locus suppresses the activity of both, the haptoglobin gene promoter as well as the herpes simplex thymidine kinase promoter in several tissues of mice (Hatada et al., J. Biol., Chem., 274(2):948-55 (1999)). Likewise, a human eNOS promoter-LacZ reporter gene placed in the Hprt locus was found to be inactive in hepatic vessels that otherwise express the endogenous eNOS gene (Guillot et al., Physiol. Genomics, Mar. 13, (2):77-83 (2000). Finally, since the HPRT gene is on the X chromosome, transgene expression at this locus is subjected to random X-inactivation. The expression of the transgene in all cells of the female, therefore, requires the generation of homozygotes.

WO 04/63381 reports on a particular autosomal locus, namely Rosa 26 that allows strong and predictable expression of transgenes inserted through homologous recombination. This chromosomal locus was found useful in the context of the “targeted transgenesis” approach for the efficient generation of transgenic organisms (such as mice) with a predictable transgene expression pattern. The “targeted transgenesis” method provided in said application comprises consecutive experimental steps. A gene expression cassette comprising a suitable promoter (e.g. a ubiquitous or tissue specific promoter, either inducible or constitutive) functionally linked to a gene of interest is created; subsequently a vector for the targeted insertion of the above mentioned gene expression cassette into the Rosa26 locus is generated; the insertion of the above mentioned gene expression cassette into the Rosa26 locus through homologous recombination or site specific recombination in embryonic stem cells follows; finally transgenic mice are generated by the injection of such genetically modified ES cells into blastocysts.

Previously, he rosa26 locus had been identified by random insertion of retroviral sequences and a β-galactosidase-neomycin resistance fusion gene into the genome of mouse embryonic stem cells (Zambrowicz et al., Proc. Natl. Acad. Sci. USA, 94, 3789-94 (1997)). The rosa26 promoter appeared to mediate ubiquitous expression of promoter-less genes both in embryos and adult mice (Kisseberth et al., Dev. Biol., 214:128-138 (1999); Zambrowicz et al., Proc. Natl. Acad. Sci. USA, 94, 3789-94 (1997)), albeit at different levels in different organs (Vooijs et al., EMBO reports, 21:292-297 (2001)).

Moreover, WO 99/53017 describes a process for making transgenic animals which ubiquitously express a heterlogous gene, wherein the heterologous gene is under the control of a ubiquitously expressed endogenous promoter, e.g. that of the mouse Rosa26 locus. R. Dacquin et al., Dev. Dynamics 224:245-251 (2002) and K. A. Moses et al., Genesis 31:176-180 (2001) utilize the transgenic mouse strain R26R obtained according to WO 99/53017 for the expression of heterlogous genes. WO 02/098217 describes a method of targeting promoter-less selection cassettes into transcriptionally active loci, such as the Rosa26 locus.

Finally, WO 03/020743 describes the expression of transgenes in vivo by targeting protected transgene cassettes into predetermined loci (e.g. the Rosa26 locus), such that the introduced tissue specific exogenous promoter has at least some tissue specific activity. The protected transgene cassette contains (from 5′ to 3′ direction) a transcriptional stop signal, the exogenous tissue specific promoter and the gene of interest. The presence of a transcriptional stop signal is vital for the method of WO 03/020743 as therewith the expression pattern is determined primarily by the nature of the tissue specific exogenous promoter.

RNA interference (RNAi) has been discovered some years ago as a tool for inhibition of gene expression (Fire, A. et al., Nature 391, 806-811 (1998)). It based on the introduction of double stranded RNA (dsRNA) molecules into cells, whereby one strand is complementary to the coding region of a target gene. Through pairing of the specific mRNA with the introduced RNA molecule, the mRNA is degraded by a cellular mechanism. Since long dsRNA provokes an interferon response in mammalian cells, the technology was initially restricted to organisms or cells showing no interferon response (Bass, B. L., Nature 411, 428-429 (2001)). The finding that short (<30 bp) interfering RNAs (siRNA) circumvent the interferon response extended the application to mammalian cells (Elbashir, S. M. et al., Nature 411, 494-498 (2001)).

Although RNAi in mice has been in principle demonstrated, the current technology does not allow performing systematic gene function analysis in vivo. So far the inhibition of gene expression has been achieved by injection of purified siRNA into the tail vain of mice (McCaffrey, A. P. et al., Nature 418, 38-39 (2002); Lewis, D. H. et al., Nature Genet. 32, 107-108 (2002)). Using this approach, gene inhibition is restricted to specific organs and persists only a few days. A further improvement of the siRNA technology is based on the intracellular transcription of short hairpin RNA (shRNA) molecules using gene expression vectors (see FIG. 1; Brummelkamp, T. R. et al., Science 296, 550-553 (2002); Paddison, P. J. et al, Genes Dev. 16, 948-958 (2002); Yu, J. Y. et al., Proc. Natl. Acad. Sci. USA 99, 6047-6052 (2002); Sui, G. et al., Proc. Natl. Acad. Sci. USA 99, 5515-5520 (2002); Paul, C. P. et al., Nature Biotechnol. 20, 505-508 (2002); Xia, H. et al., Nat. Biotechnol. 10, 1006-10 (2002); Jacque, J. M. et al., Nature 418(6896):435-8 (2002)). The activity of shRNA in mice has been demonstrated by McCaffrey et al., 2002 through injection of shRNA expression vectors into the tail vain. Again, gene inhibition was temporally and spatially restricted. Although these results demonstrate that the mechanism of shRNA mediated gene silencing is functional in mice, they do not clarify whether constitutive RNAi can be achieved in transgenic animals. Brummelkamp, T. R. et al., Science 296, 550-553 (2002), Paddison, P. J. et al., Genes Dev. 16, 948-958 (2002), Hemann, M. T. et al., Nat. Genet. 33(3):396-400 (2003); and Devroe, E. et al., BMC Biotechnol. 2(1):15 (2002) have shown the long-term inhibition of gene expression through stable integration of shRNA vectors in cultivated cell lines. These experiments included random integration of shRNA transgenes and screening for clones with appropriate siRNA expression, which is not applicable for testing of a large number of different shRNA transgenes in mice. Finally, several reports have demonstrated shRNA-mediated gene silencing in transgenic mice and rats (Hasuwa, H. et al., FEBS Lett. 532(1-2):227-30 (2002); Carmell, M. A. et al., Nat. Struct. Biol. 10(2):91-2 (2003); Rubinson, D. A. et al., Nat. Genet. 33(3):401-6 (2003); Kunath, T. et al., Nat. Biotechnol. (Apr. 7, 2003)). However, these experiments again included random integration of shRNA transgenes resulting in variable levels and patterns of shRNA expression. Thus, testing of ES cell clones or mouse lines with appropriate shRNA expression had been required, which is a laborious and time-consuming undertaking.

The in vivo validation of genes by RNAi mediated gene repression in a large scale setting requires the expression of siRNA at sufficiently high levels and with a predictable pattern in multiple organs. Targeted transgenesis provides the only approach to achieve reproducible expression of transgenes in the living organism (e.g. mammalians such as mice). WO 04/035782 discloses for the first time that a single copy of a siRNA expression vector integrated into a defined locus of the genome can provide sufficiently high levels of siRNA for efficient RNAi-mediated gene inhibition in multiple organs of the living organism.

Two types of procedures have been described for targeted integration of transgenes into defined loci of the embryonic stem (ES) cell genome. One is based on homologous recombination (HR) in embryonic stem cells, and the other on site-specific recombination. In the first case, the efficiency is limited by the low frequency of HR. In contrast, site-specific recombination has emerged as a powerful tool for the targeted insertion of transgenes into the eukaryotic genome.

Site-specific recombinases such as Flp and Cre mediate recombination between two copies of their target sequence termed FRT and loxP, respectively. The use of two incompatible target sequences, for example FRT in combination with F3 (Schlake & Bode, Biochemistry, 1994 Nov. 1, 33(43):12746-51) as well as inverted recognition target, sites (Feng et al., J. Mol. Biol. 292(4):779-85 (1999)) allows the insertion of DNA segments into a predefined chromosomal locus carrying target sequences in a similar configuration. This exchange system is called recombinase mediated cassette exchange (RMCE; Bode & Baer, Curr Opin Biotechnol. 2001 October; 12(5):473-80). In contrast to approaches using a single recombination site the targeting product is stable even under the permanent influence of the recombinase unless it is exposed to an exchange plasmid (Seibler & Bode (1997) Biochemistry 36, 1740-1747.).

So far, only few examples of successful RMCE in ES have been described (Feng et al., 3 Mol. Biol. 1999 Oct. 1; 292(4):779-85; Seibler et al., Biochemistry. 1998 May 5; 37(18):6229-34; Kolb, Anal Biochem. 2001 Mar. 15; 290(2):260-71; Belteki et al., Nat. Biotechnol. 2003 March; 21(3):321-4.; Cesari et al., 2004, Genesis, 38:87-92.). In these experiments, random integration of the exchange vector as well as incomplete recombination frequently produced unwanted transgene configurations. The efficiency of RMCE appeared to vary strongly depending on the choice of recombination sites, the selection strategy, and the chromosomal target. The criteria for efficient RMCE at a given locus are therefore not defined and unpredictable for a person skilled in the art.

The only example of efficient (>90%) RMCE at a defined locus used the tissue-specific β-casein gene as chromosomal target (Kolb, Anal Biochem. 2001 Mar. 15; 290(2):260-71). However, a HPRT gene was required to exclude random integration or incomplete recombination of the exchange vector. The application of this strategy is therefore limited to HPRT-negative ES cells. In addition, the cell type specific activity of the B-Casein locus may not be suitable for the expression of transgenes in multiple tissues. Taken together, a general strategy for efficient RMCE at a ubiquitously active locus has never been achieved.

### SUMMARY OF THE INVENTION

It was surprisingly found that RMCE can be effectively be performed at ubiquitously active loci with high efficiency. The invention provides:

(1) a method for generating transgenic eukaryotic cells having an ubiquitous locus modified by an expression cassette comprising a short hairpin RNA construct operatively linked to a promoter or an inactive precursor thereof, which method comprises introducing the expression cassette into the ubiquitous locus of eukaryotic cells by recombinase mediated cassette exchange;
(2) the method of (1) above, which comprises
(a) introducing a functional DNA sequence into the Rosa26 locus of starting eukaryotic cells by homologous recombination with a targeting vector comprising flanking DNA sequences homologous to the ubiquitous locus and an acceptor DNA, which integrates into the genome of the starting cell, the acceptor DNA comprising two mutally incompatible first recombinase recognition sites (RRSs), and
(b) effecting recombinase mediated cassette exchange of the recombination product of step (a) having an RMCE target site with an exchange vector comprising a donor DNA, which comprises the expression cassette flanked by the same two mutually incompatible first RRSs contained in the acceptor DNA, by utilizing a recombinase which catalyzes recombination between the RRSs of the acceptor and donor DNA; (3) the method of (1) or (2) above, wherein the transgenic eukaryotic cells are derived from mouse and the ubiquitous locus is a Rosa26 locus, and
(i) the DNA sequences homologous to the Rosa26 locus are derived from the 5′ and 3′ flanking arm of the mouse Rosa26 locus, preferably said homologous DNA sequences having the sequences shown in SEQ ID NO:4 and 5, respectively, and/or
(ii) the RRSs of the targeting and exchange vectors are F3/Frt and the targeting vectors encodes the recombinase Flp or a mutant thereof, preferably Flpe; and/or
(iii) the targeting vector comprises a negative selection marker; and/or
(iv) the exchange vector comprises a promoter-less positive selection marker; and/or
(v) the promoter of the expression cassette is a H1 or H6; most preferably the targeting vector has the sequence shown in SEQ ID NO:11 and the exchange vector has the sequence shown in SEQ ID NO: 12 or a variant thereof with modification in the short hairpin RNA construct;
(4) an exchange vector as defined in (1) to (3) above;
(5) a eukaryotic cells having a modified ubiquitous locus obtainable by the method of (1), (2) and (3) above;
(6) a method for preparing a transgenenic multi-cell organism having a modified ubiquitous locus which comprises utilizing the method as defined in (1) and (3) above;
(7) the method of (6) above, wherein the transgenenic multi-cell organism is a non-human mammal and said method comprises modifying an ES cell as defined in (3) above;
(8) a transgenic multi-cell organism and non-human mammal obtainable by the above defined methods (6) and (7), respectively; and
(9) the use of the eukaryotic cell of (5) above, the transgenic multi-cell organism of (8) above, or the transgenic non-human mammal of (8) above for gene function studies, drug development, as disease model, etc.

The method of the invention offers several advantages over the current technology of pronuclear injection. In particular, the targeting vector allows insertion of a single copy of a gene expression cassette, thus avoiding modulation of transgene expression by the arrangement of multiple copies. By choosing the autosomal Rosa26 locus as insertion site, the expression pattern of the inserted transgene in the non-human animal is predictable; random X-inactivation and/or modulation by chromosomal position effects are avoided. This also eliminates the need to generate and analyse multiple transgenic strains for any given transgene. Finally, the Rosa26 targeting vector for the site-specific integration can be used for multiple gene expression cassettes. Moreover, the RMCE strategy provides for more flexibility for consitutive and inducible gene knock-down, RNA mediated gene silencing in transgene animals and living organs.

### DESCRIPTION OF THE FIGURES

FIG. 1: Targeted insertion of CreER and CAGGS-Cre-ER into the Rosa26 locus. A cassette comprising a Cre-ER operationally linked to a CAGGS promoter or a cassette comprising a splice acceptor site (SA) linked to a Cre-ER are inserted into the Rosa26 locus via homologous recombination. A perpendicular dash marks the insertion point within the Rosa26 locus and the rectangular boxes delinate the starting and end points of the Rosa26 transcript.

FIG. 2: Southern Blot analysis of the inducible recombination of the Rosa (reporter). (A) Genomic DNA was isolated from liver (Li) spleen (Sp) and small intestine (Si) of transgenic mice carrying the SA-creER/Rosa-rep insert or the CAGGS-creER/Rosa-rep insert. To induce the Cre-ER recombinase the mice were treated with Tamoxifen (treated). As a control, a group of mice with the SA-creER/Rosa-rep insert was left untreated (untreated). Presence of the reporter band (floxed) and deletion (deleted) of it upon an induced recombination event are indicated. (B) Transgenic mice carrying at one Rosa26 locus a loxP flanked DNA polymerase β gene segment (pol^{βflox}) and at the other a SA-creER/Rosa-rep were treated with Tamoxifen (treated). A control group of mice was left untreated (untreated). Genomic DNA from liver (Li), spleen (Sp), kidney (Ki). heart (He), lung (Lu), thymus (Th), muscle (Mu), small intestine (Si) and brain (Br) was analysed for presence of pol^{βflox}. In a non-recombination event the pol^{βflox} band remained (floxed), in a recombination event deletion occurred (deleted). (C) As (B), but mice carried instead of the SA-creER/Rosa-rep the CAGGS-creER/Rosa-rep insert.

FIG. 3: Western Blot analysis of recombinase and α-actin expression. Proteins were extracted from rosa(SA-CreER^{T2}) and rosa (CAGGS-CreER^{T2}) mice and analyzed as described in the “Materials and Method” section. The positions of bands representing CreERn and actin are indicated. FA: fat tissue, Ty: Thymus; Sp: spleen, Br: Brain, Lu: lung, He: heart.

FIG. 4: Fabp-Cre targeting vector. An expression cassette, in which the Cre recombinase is expressed under the control of the Fabpl^{4x at -132} promoter is inserted into the Rosa26 targeting vector. This vector was used to insert the Fabp-Cre cassette into the Rosa26 locus by homologous recombination in ES cells.

FIG. 5: ROSA26 locus of the Cre reporter mice carrying a Cre substrate reporter construct. A recombination substrate (Seq ID NO:9) has been inserted in the ROSA26 locus. The substrate consists of a CAGGS promoter followed by a cassette consisting of the hygromycin resistance gene driven by a PGK promoter and flanked by loxP recombination sites. This cassette is followed by the coding region for beta-galactosidase, which is only expressed when the hygromycin resistance gene has been deleted by recombination.

FIG. 6: In situ detection of beta-galactosidase in cryosections of different tissues of Fabp-Cre/reporter substrate double transgenic mice. Mouse tissues were embedded in OCT, frozen and cut into microsections. The sections were stained for beta-galactosidase activity (indicated by the blue color) by X-gal staining, counterstained with Nuclear Fast Red Solution, dehydrated, mounted and photographed.

FIG. 7: RMCE targeting system for rosa26. A) Insertion of the RMCE target into the rosa26 locus. A cassette comprising zsgreen, PGK-Hyg, and CAGGS-FLP is inserted into the Rosa26 locus via homologous recombination in ES cells. The FRT and F3 sites are oriented in opposite direction to each other. A perpendicular dash with ‘X’ marks the insertion point within the rosa26 locus. B) Exchange vector carrying FRT and F3 sites together with a truncated neo^{R} gene for positive selection of RMCE and a shRNA expression cassette under the control of the U6 promoter for targeted integration into the Rosa26 locus. The polyA signal is included to prevent expression of the truncated neo^{R} gene at sites of random integration. C) Configuration of the targeted Rosa26 locus following. X: XbaI, H: HindIII.

FIG. 8: Southern blot analysis of genomic DNA from rosa(RMCE) targeted ES cells transfected with the exchange vector. rosa(RMCE exchanged) alleles. The sizes of the wt. Rosa26 targeted (10 HR) and RMCE alleles (exchange) are 4.4 kb, 3.9 kb and 5.8 kb, respectively. In clones #1-3, 5-9, and 11-16 successful RMCE had occurred. Genomic DNA was digested with HindIII and analyzed using probe 1.

### DETAILED DESCRIPTION OF THE INVENTION

The term “living organisms” according to the present invention relates to multi-cell organisms which can be vertebrates such as mammals (e.g. non-human animals such as rodents including mice and rats; and humans) or non-mammals (e.g. fish) or can be invertebrates such as insects or worms, or can be plants (higher plants, algi or fungi). Most preferred living organisms are mice and fish.

“Eukaryotic cells” and “starting eukaryotic cells” according to the present invention include cells isolated (derived) from the above defined living organisms and cultured in vitro. These cells can be transformed (immortalized) or untransformed (directly derived from living organisms; primary cell culture). The term “eukaryotic cells” also includes mono-cellular eukaryqtic cells such as yeasts, etc.

It is preferred in the method (1) of the present invention that the eukaryotic cells are derived from a multi-cell organism including vertebrates, invertebrates and plants, preferably is a vertebrate cell, more preferably is derived from a mammal, including rodents such as mouse, rat, etc., or a fish such as zebrafish.

In the method (1) of the invention it is preferred that the functional DNA sequence comprises a gene encoding a protein/peptide of interest (i.e. is a expressible and translatable DNA sequence), more preferably said functional DNA sequence is a gene expression cassette (a) comprising a gene of interest operatively linked to a promoter, or (b) is a DNA sequence which can be converted into such gene expression cassette (i.e. into an operatively linked “promoter-gene of interest” construct, e.g. by subsequent modification reactions after its integration). The gene of interest within the gene expression cassette can be any gene coding for a certain protein/peptide of interest, including, but not limited to, recombinases, reporter genes, receptors, signaling molecules, transcription factors, pharmaceutically active proteins and peptides, drug target candidates, disease causing gene products, toxins, etc.

The promoter of the gene expression cassette (which is a heterologous promoter relative to the Rosa26 locus) preferably is a ubiquitous or tissue specific promoter, either constitutive or inducible. The ubiquitous promoter in the vector according to the invention is preferably selected from polymerases I, II and III dependent promoters, preferably is a polymerase II or III dependent promoter including, but not limited to, a CMV promoter, a CAGGS promoter, a snRNA promoter such as U6, a RNAse P RNA promoter such as H1, a tRNA promoter, a 7SL RNA promoter, a 5 S rRNA promoter, etc. Particularly preferred ubiquitous promoters are CAGGS, hCMV, PGK. Preferred tissue specific promoters are FABP (Saam & Gordon, J. Biol. Chem., 274:38071-38082 (1999)), Lck (Orban et al., Proc. Natl. Acad. Sci. USA, 89:6861-5 (1992)), CamKII (Tsien et al., Cell 87: 1317-1326 (1996)), CD19 (Rickert et al., Nucleic Acids Res. 25:1317-1318 (1997)), Keratin (Li et al., Development, 128:675-88 (201)), Albumin (Postic & Magnuson, Genesis, 26:149-150 (2000)), aP2 (Barlow et al., Nucleic Acids Res., 25 (1997)), Insulin (Ray et al., Int. J. Pancreatol. 25:157-63 (1999)), MCK (Bruning et al., Molecular Cell 2:559-569 (1998)), MYHC (Agak et al., J. Clin. Invest., 100:169-179 (1997), WAP (Utomo et al., Nat. Biotechnol. 17:1091-1096 (1999)), Col2A (Ovchinnikov et al., Genesis, 26:145-146 (2000)); preferred inducible promoter systes are Mx (Kuhn et al. Scinence, 269:1427-1429 (1995)), tet (Urlinger et al., Proc. Natl. Acad. Sci. USA, 97:7963-8 (2000)), Trex (Feng and Erikson, Human Gene Therapy, 10:419-27). Suitable inducible promoters are the above-mentioned promoters containing an operator sequence including, but not limited to, tet, Gal4, lac, etc.

The targeting vector, recombination vector, functional DNA sequence or gene expression cassette may further comprises one or more additional functional sequences including but not limited to (selectable) marker genes (such as the neomycin phosphotransferase gene of E. coli transposon, etc.), recombinase recognition sites (which in case of the recombination vector differ from the first recombinase recognition sites and which include loxP, FRT, variants thereof, etc.), poly A signals (such as synthetic polyadenylation sites, or the polyadenylation site of human growth hormones, etc.), splice acceptor sequences (such as a splice acceptor of adenovirus, etc.), introns, tags for protein detection, enhancers, selection markers, etc.

In a preferred embodiment methods (1) to (3) of the invention comprise homologous recombination. It is then preferred that the DNA sequences homologous to the Rosa26 locus are 0.2 to 20 kB, preferably 1 to 10 kB long. In a particularly preferred embodiment of the method (2) the eukaryotic cells are derived from mouse, the DNA sequences homologous to the Rosa26 locus are derived from the 5′ and 3′ flanking arm of the mouse Rosa26 locus, preferably said homologous DNA sequences having the sequences shown in SEQ ID NO:4 and 5, respectively, and the promoter is a CAGGS-promoter, most preferably the targeting vector has the sequence shown in SEQ ID NO:7.

As set forth above, methods (1) to (3) of the invention comprise recombinase mediated cassette exchange (RMCE). The insertion of transgenes or DNA segments into the genome can be mediated by site specific recombination (Fukushige & Sauer, Proc. Natl. Acad. Sci. USA 89(17):7905-9 (1992)). A site specific recombinase like cre or FLP recombines two recognition target sites like loxP or FRT, respectively. The use of two incompatible recognition target sites (F3 or F5, Schlake & Bode, Biochemistry, 1994 Nov. 1, 33(43):12746-51) or inverted recognition target sites (Feng et al., J. Mol. Biol. 292(4):779-85 (1999)) allows the insertion of DNA segments flanked by two incompatible or inverted target sites. This exchange system has been called recombinase mediated cassette exchange (RMCE). In a preferred embodiment a FLP based RMCE system is inserted into the Rosa26 locus. Said recombinase mediated recombination preferably comprises the steps:
(a1) introducing into the starting cells an acceptor DNA which integrates into the genome of the starting cell, the acceptor DNA comprising two mutally incompatible first RRSs, and introducing into the therewith obtained cell
(a2) a donor DNA comprising the same two mutually incompatible first RRSs contained in the acceptor DNA by utilizing a recombination vector as defined above; and
(a3) the recombinase which catalyzes recombination between the RRSs of the acceptor and donor

In said recombinase mediated recombination method it is preferred that
(i) the RRS are loxP or FRT sites or variants thereof (such as single mutant recognition sited lox66 and lox71 (Albert et al., The Plant 3. 7:649-659 (1995)); and/or
(ii) the acceptor DNA comprises a negatively selectable marker (e.g. herpes simplex virus thymidin kinase gene, etc.) and or
(iii) the donor DNA comprises an inactive positive selection marker (e.g. neomycin phosphotransferase, etc.).

For further selectable markers it is referred to U.S. Pat. Nos. 5,487,932 and 5,464,763 which are hereby incorporated in their entirety.

The ubiquitous promoter in the vector according to the invention is preferably selected from polymerase I, II and III dependent promoters, preferably is a polymerase II or III dependent promoter including, but not limited to, a CMV promoter, a CAGGS promoter, a snRNA promoter such as U6, a RNAse P RNA promoter such as H1, a tRNA promoter, a 7SL RNA promoter, a 5 S rRNA promoter, etc.

The ubiquitous promoter can be a constitutive promoter, or can be an inducible promoter. Suitable inducible promoters are the above-mentioned polymerase I, II and III dependent promoters containing an operator sequence including, but not limited to, tet, Gal4, lac, etc.

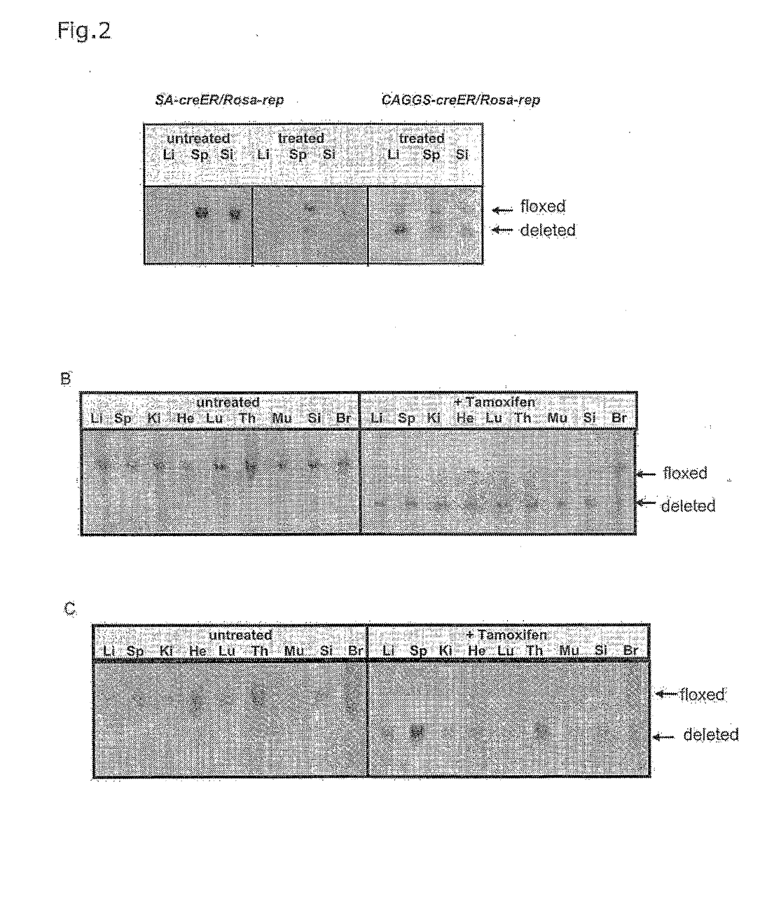

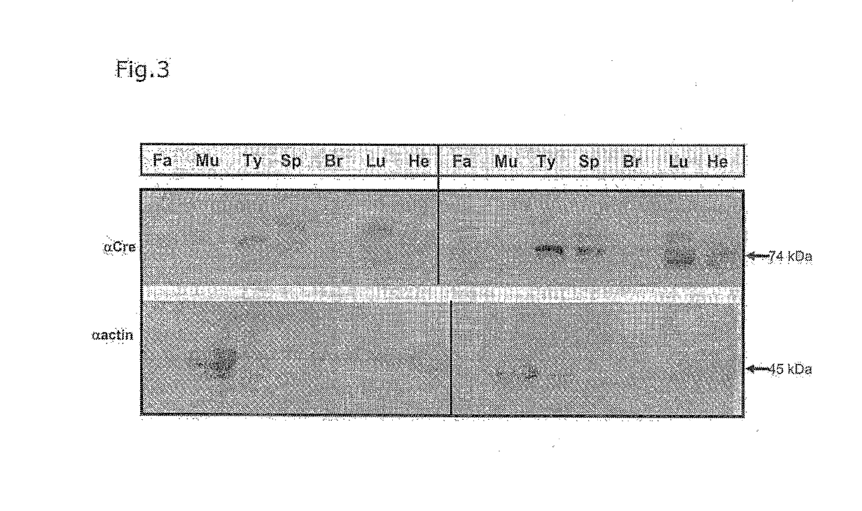

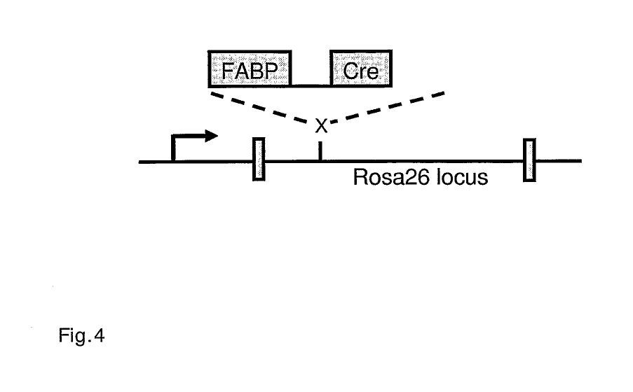

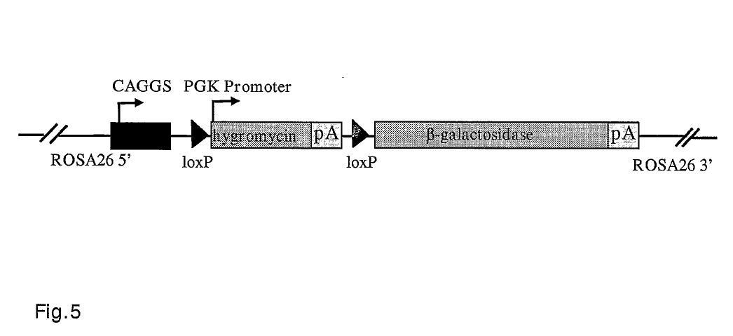

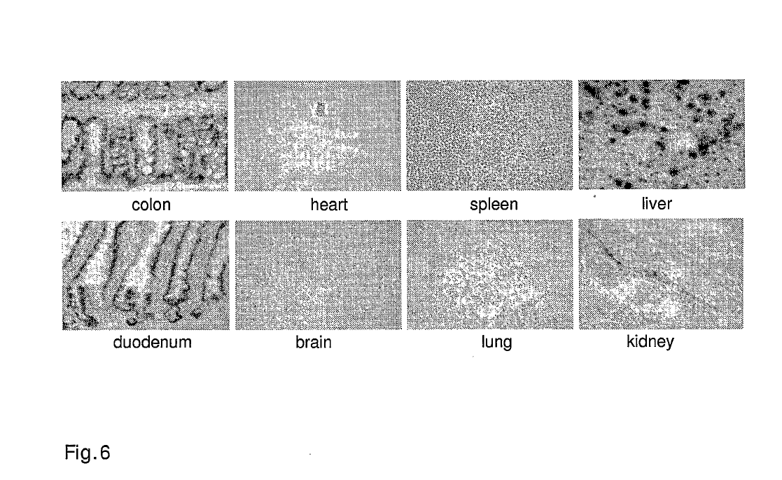

The expression vector of the invention is suitable for the following particularly preferred approaches (for constitutive and inducible expression):
A. a Pol III dependent promoter (constitutive U6, H1 or the like) driven shRNA construct (to be integrated into a ubiquitously active Pol II dependent locus (see FIG. 2);
B. a Pol III dependent promoter (inducible U6, H1 or the like) driven shRNA construct (to be integrated into a ubiquitously active Pol II dependent locus (FIGS. 3 and 4)); or
C. a polymerase II (Pol II) dependent promoter (inducible CMV or the like) driven shRNA construct (to be integrated into a ubiquitously active Pol II dependent locus (FIGS. 5 and 6)).

The short hairpin RNA construct or inactive precursor thereof of the expression cassette comprises at least one segment corresponding to a short hairpin RNA (shRNA) or to complementary short interfering RNA (siRNA) strands. In case shRNA segments are utilized within the expression cassette, said cassette preferably comprises at least one shRNA segment having a nucleotide (e.g. DNA) sequence of the structure A-B-C or C-B-A. In case siRNA segments are utilized within the expression cassette, said cassette preferably comprises at least least two DNA segments A and C or C and A, wherein each of said at least two segments is under the control of a separate promoter as defined above (such as the Pol III promoter including inducible U6, H1 or the like). In the above segments
A is a 15 to 35, preferably a 19 to 29 bp DNA sequence being at least 90%, preferably 100% complementary to the gene to be knocked down (e.g. firefly luciferase, p53, etc.);
B is a spacer DNA sequence having 5 to 9 bp forming the loop of the expressed RNA hairpin molecule, and
C is a 15 to 35, preferably a 19 to 29 bp DNA sequence being at least 85% complementary to the sequence A.

The above shRNA and siRNA segments may further comprise stop and/or polyadenylation sequences.

Suitable shRNA sequences for the knock down of a given target gene are well known in the art (see e.g. the particular shRNA sequences mentioned in Tables 1 and 2 below) or can readily be determined by the skilled artesian.

TABLE 1

| target | | |
|---|---|---|
| gene | shRNA sequence/SEQ ID NO | Reference |
| CDH-1 | TgagaagtctcccagtcagTTCAAGAGActgactgggagacttctca (13) | Brummelkamp |
| p53 | GactccagtggtaatctacTTCAAGAGAgtagattaccactggagtc (14) | et al., |
| CDC20 | CggcaggactccgggccgaTTCAAGAGAtcggcccggagtcctgccg (15) | Science, 296: |
| | | 550-3 (2002). |
| CYLD | CctcatgcagttctctttgTTCAAGAGAcaaagagaactgcatgagg (16) | Kovalenko et |
| | | al, Nature, |
| | | 424:801-5 |
| | | (2003). |
| Ras- | AagatgaagccactccctatttCAAGAGAaaatagggagtggcttcatctt | Kunath et al., |
| Gap | (17) | Nature |
| | | Biotechnology, |
| | | 21:559-561 |
| | | (2003). |
| tubulin | GacagagccaagtggactcACAgagtccacttggctctgtc (18) | Yu et al., |
| | | PNAS, 99: |
| | | 6047-52 |
| | | (2002) |
| lamin | Ctggacttccagaagaacattcgtgttcttctggaagtccag (19) | Paul et al., |
| | | Nature Bio- |
| | | technology, |
| | | 20:505-8 |
| | | (2002). |

TABLE 2

shRNA sequences known from Brummelkamp et al., Nature,
424:797-801 (2003):

| Target | |
|---|---|
| Gene | shRNA Sequence/SEQ ID NO |
| UBIQUITIN | GAGATTGGTCCAGAACAGTTTCAAGAGAACTGTTCTGGACCAATCTC (20) |
| CARBOXYL- | GCCCTTCCGATCATGGTAGTTCAAGAGACTACCATGATCGGAAGGGC (21) |
| TERMINAL | TCTTTAGAATTCTTAAGTATTCAAGAGATACTTAAGAATTCTAAAGA (22) |
| HYDROLASE 12 | CATTAGCTATATCAACATGTTCAAGAGACATGTTGATATAGCTAATG (23) |
| UBIQUITIN | ACCACAAACGGCGGAACGATTCAAGAGATCGTTCCGCCGTTTGTGGT (24) |
| CARBOXYL- | GAGGGTCTTGGAGGTCTTCTTCAAGAGAGAAGACCTCCAAGACCCTC (25) |
| TERMINAL | GTCCATGCCCAGCCGTACATTCAAGAGATGTACGGCTGGGCATGGAC (26) |
| HYDROLASE 11 | GCTGGACACCCTCGTGGAGTTCAAGAGACTCCACGAGGGTGTCCAGC (27) |
| UBIQUITIN | GAATATCAGAGAATTGAGTTTCAAGAGAACTCAATTCTCTGATATTC (28) |
| CARBOXYL- | TGGACTTCATGAGGAAATGTTCAAGAGACATTTCCTCATGAAGTCCA (29) |
| TERMINAL | TATTGAATATCCTGTGGACTTCAAGAGAGTCCACAGGATATTCAATA (30) |
| HYDROLASE 10 | TTGTACTGAGAGAAACTGCTTCAAGAGAGCAGTTTCTCTCAGTACAA (31) |
| HAUSP | GATCAATGATAGGTTTGAATTCAAGAGATTCAAACCTATCATTGATC (32) |
| | GGAGTTTGAGAAGTTTAAATTCAAGAGATTTAAACTTCTCAAACTCC (33) |
| | GAACTCCTCGCTTGCTGAGTTCAAGAGACTCAGCAAGCGAGGAGTTC (34) |
| | CCGAATTTAACAGAGAGAATTCAAGAGATTCTCTCTGTTAAATTCGG (35) |
| UBIQUITIN | GACAGCAGAAGAATGCAGATTCAAGAGATCTGCATTCTTCTGCTGTC (36) |
| CARBOXYL- | ATAAAGCTCAACGAGAACCTTCAAGAGAGGTTCTCGTTGAGCTTTAT (37) |
| TERMINAL | GGTGAAGTGGCAGAAGAATTTCAAGAGAATTCTTCTGCCACTTCACC (38) |
| HYDROLASE 8 | GTATTGCAGTAATCATCACTTCAAGAGAGTGATGATTACTGCAATAC (39) |
| FLJ10785 | GATATGGGGTTCCATGTCATTCAAGAGATGACATGGAACCCCATATC (40) |
| | GGAGACATGGTTCTTAGTGTTCAAGAGACACTAAGAACCATGTCTCC (41) |
| | AGCACCAAGTTCGTCTCAGTTCAAGAGACTGAGACGAACTTGGTGCT (42) |
| | GATGCAACACTGAAAGAACTTCAAGAGAGTTCTTTCAGTGTTGCATC (43) |
| KIAA0710 | GTCAATGGCAGTGATGATATTCAAGAGATATCATCACTGCCATTGAC (44) |
| | CCTGCTAGCTGCCTGTGGCTTCAAGAGAGCCACAGGCAGCTAGCAGG (45) |
| | CCACCTTTGCCAGAAGGAGTTCAAGAGACTCCTTCTGGCAAAGGTGG (46) |
| | CCCTATTGAGGCAAGTGTCTTCAAGAGAGACACTTGCCTCAATAGGG (47) |
| FLJ12552/ | GAAGGAAAACTTGCTGACGTTCAAGAGACGTCAGCAAGTTTTCCTTC (48) |
| FLJ14256 | CTCACCTGGGTCCATGAGATTCAAGAGATCTCATGGACCCAGGTGAG (49) |
| | GCTGTCTTACCGTGTGGTCTTCAAGAGAGACCACACGGTAAGACAGC (50) |
| | CCTGGACCGCATGTATGACTTCAAGAGAGTCATACATGCGGTCCAGG (51) |
| KIAA1203 | GTCAATGGCAGTGATGATATTCAAGAGATATCATCACTGCCATTGAC (52) |
| | CCTGCTAGCTGCCTGTGGCTTCAAGAGAGCCACAGGCAGCTAGCAGG (53) |
| | CCACCTTTGCCAGAAGGAGTTCAAGAGACTCCTTCTGGCAAAGGTGG (54) |
| | CCCTATTGAGGCAAGTGTCTTCAAGAGAGACACTTGCCTCAATAGGG (55) |
| FLJ23277 | GGAAATCCGAATTGCTTGGTTCAAGAGACCAAGCAATTCGGATTTCC (56) |
| | CACATTTCTTCAAGTGTGGTTCAAGAGACCACACTTGAAGAAATGTG (57) |
| | CAGCAGGATGCTCAAGAATTTCAAGAGAATTCTTGAGCATCCTGGTG (58) |
| | GCTGAATACCTACATTGGCTTCAAGAGAGCCAATGTAGGTATTCAGC (59) |
| FLJ14914 | GGGCTTGTGCCTGGCCTTGTTCAAGAGACAAGGCCAGGCACAAGCCC (60) |
| (similar | GCCTTGTCCTGCCAAGAAGTTCAAGAGACTTCTTGGCAGGACAAGGC (61) |
| to UBP4) | GATTGAAGCCAAGGGAACGTTCAAGAGACGTTCCCTTGGCTTCAATC (62) |
| | TGGCGCCTGCTCCCCATCTTTCAAGAGAAGATGGGGAGCAGGCGCCA (63) |
| UBIQUITIN | GAACCAGCAGGCTCTGTGGTTCAAGAGACCACAGAGCCTGCTGGTTC (64) |
| CARBOXYL- | GGAAGCATAATTATCTGCCTTCAAGAGAGGCAGATAATTATGCTTCC (65) |
| TERMINAL | AGAAGAAGATGCTTTTCACTTCAAGAGAGTGAAAAGCATCTTCTTCT (66) |
| HYDROLASE | CTTGCAGAGGAGGAACCCATTCAAGAGATGGGTTCCTCCTCTGCAAG (67) |
| ISOZYME L5 | |
| UBIQUITIN | GCAAACAATCAGCAATGCCTTCAAGAGAGGCATTGCTGATTGTTTGC (68) |
| CARBOXYL- | TTGGACTGATTCATGCTATTTCAAGAGAATAGCATGAATCAGTCCAA (69) |
| TERMINAL | CTGGCAATTCGTTGATGTATTCAAGAGATACATCAACGAATTGCCAG (70) |
| HYDROLASE | TTAGATGGGCGGAAGCCATTTCAAGAGAATGGCTTCCGCCCATCTAA (71) |
| ISOZYME L3 | |
| UBIQUITIN | GAGGAGTCTCTGGGCTCGGTTCAAGAGACCGAGCCCAGAGACTCCTC (72) |
| CARBOXYL- | GAGCTGAAGGGACAAGAAGTTCAAGAGACTTCTTGTCCCTTCAGCTC (73) |
| TERMINAL | TGTCGGGTAGATGACAAGGTTCAAGAGACCTTGTCATCTACCCGACA (74) |
| HYDROLASE | CACAGCTGTTCTTCTGTTCTTCAAGAGAGAACAGAAGAACAGCTGTG (75) |
| ISOZYME L1 | |
| KIAA1891/ | GTGGAAGCCTTTACAGATCTTCAAGAGAGATCTGTAAAGGCTTCCAC (76) |
| FLJ25263 | CAACAGCTGCCTTCATCTGTTCAAGAGACAGATGAAGGCAGCTGTTG (77) |
| | CCATAGGCAGTCCTCCTAATTCAAGAGATTAGGAGGACTGCCTATGG (78) |
| | TGTATCACTGCCACTGGTTTTCAAGAGAAACCAGTGGCAGTGATACA (79) |
| FLJ14528 | CATGTTGGGCAGCTGCAGCTTCAAGAGAGCTGCAGCTGCCCAACATG (80) |
| (similar | CACAACTGGAGACCTGAAGTTCAAGAGACTTCAGGTCTCCAGTTGTG (81) |
| to UBP8) | GTATGCCTCCAAGAAAGAGTTCAAGAGACTCTTTCTTGGAGGCATAC (82) |
| | CTTCACAGTACATTTCTCTTTCAAGAGAAGAGAAATGTACTGTGAAG (83) |
| U4/U6 TRI | GTACTTTCAAGGCCGGGGTTTCAAGAGAACCCCGGCCTTGAAAGTAC (84) |
| SNRNP 65 kDa | CTTGGACAAGCAAGCCAAATTCAAGAGATTTGGCTTGCTTGTCCAAG (85) |
| protein | GACTATTGTGACTGATGTTTTCAAGAGAAACATCAGTCACAATAGTC (86) |
| | GGAGAACTTTCTGAAGCGCTTCAAGAGAGCGCTTCAGAAAGTTCTCC (87) |
| XM_089437 | GACGAGAGAAACCTTCACCTTCAAGAGAGGTGAAGGTTTCTCTCGTC (88) |
| | ACATTATTCTACATTCTTTTTCAAGAGAAAAGAATGTAGAATAATGT (89) |
| | AGATTCGCAAATGGATGTATTCAAGAGATACATCCATTTGCGAATCT (90) |
| | CATTCCCACCATGAGTCTGTTCAAGAGACAGACTCATGGTGGGAATG (91) |
| KIAA1453 | GATCGCCCGACACTTCCGCTTCAAGAGAGCGGAAGTGTCGGGCGATC (92) |
| | CCAGCAGGCCTACGTGCTGTTCAAGAGACAGCACGTAGGCCTGCTGG (93) |
| | GCCAGCTCCTCCACAGCACTTCAAGAGAGTGCTGTGGAGGAGCTGGC (94) |
| | CGCCGCCAAGTGGAGCAGATTCAAGAGATCTGCTCCACTTGGCGGCG (95) |
| FLJ12697 | GAAGATGCCCATGAATTCCTTCAAGAGAGGAATTCATGGGCATCTTC (96) |
| | CAAACAGGCTGCGCCAGGCTTCAAGAGAGCCTGGCGCAGCCTGTTTG (97) |
| | ACGGCCTAGCGCCTGATGGTTCAAGAGACCATCAGGCGCTAGGCCGT (98) |
| | CTGTAACCTCTCTGATCGGTTCAAGAGACCGATCAGAGAGGTTACAG (99) |
| UBIQUITIN | TCTGTCAGTCCATCCTGGCTTCAAGAGAGCCAGGATGGACTGACAGA (100) |
| SPECIFIC | TGAAGCGAGAGTCTTGTGATTCAAGAGATCACAAGACTCTCGCTTCA (101) |
| PROTEASE 18 | GATGGAGTGCTAATGGAAATTCAAGAGATTTCCATTAGCACTCCATC (102) |
| (USP18) | CCTTCAGAGATTGACACGCTTCAAGAGAGCGTGTCAATCTCTGAAGG (103) |
| UBIQUITIN | CCTGACCACGTTCCGACTGTTCAAGAGACAGTCGGAACGTGGTCAGG (104) |
| CARBOXYL- | GAGTTCCTTCGCTGCCTGATTCAAGAGATCAGGCAGCGAAGGAACTC (105) |
| TERMINAL | GACTGCCTTGCTGCCTTCTTTCAAGAGAAGAAGGCAGCAAGGCAGTC (106) |
| HYDROLASE 20 | CGCCGAGGGCTACGTACTCTTCAAGAGAGAGTACGTAGCCCTCGGCG (107) |
| UBIQUITIN | GGCGAGAAGAAAGGACTGTTTCAAGAGAACAGTCCTTTCTTCTCGCC (108) |
| CARBOXYL- | GGACGAGAATTGATAAAGATTCAAGAGATCTTTATCAATTCTCGTCC (109) |
| TERMINAL | GCACGAGAATTTGGGAATCTTCAAGAGAGATTCCCAAATTCTCGTGC (110) |
| HYDROLASE 24 | CTACTTCATGAAATATTGGTTCAAGAGACCAATATTTCATGAAGTAG (111) |
| KIAA1594 | GATAACAGCTTCTTGTCTATTCAAGAGATAGACAAGAAGCTGTTATC (112) |
| | GAGAATAGGACATCAGGGCTTCAAGAGAGCCCTGATGTCCTATTCTC (113) |
| | CTTGGAAGACTGAACCTGTTTCAAGAGAACAGGTTCAGTCTTCCAAG (114) |
| | CAACTCCTTTGTGGATGCATTCAAGAGATGCATCCACAAAGGAGTTG (115) |
| KIAA1350 | GATGTTGTCTCCAAATGCATTCAAGAGATGCATTTGGAGACAACATC (116) |
| | CGTGGGGACTGTACCTCCCTTCAAGAGAGGGAGGTACAGTCCCCACG (117) |
| | GTACAGCTTCAGAACCAAGTTCAAGAGACTTGGTTCTGAAGCTGTAC (118) |
| UBIQUITIN | GATGATCTTCAGAGAGCAATTCAAGAGATTGCTCTCTGAAGATCATC (119) |
| CARBOXYL- | GGAACATCGGAATTTGCCTTTCAAGAGAAGGCAAATTCCGATGTTCC (120) |
| TERMINAL | GAGCTAGTGAGGGACTCTTTTCAAGAGAAAGAGTCCCTCACTAGCTC (121) |
| HYDROLASE 25 | GCAGGGTTCTTTAAGGCAATTCAAGAGATTGCCTTAAAGAACCCTGC (122) |
| UBIQUITIN | TCGATGATTCCTCTGAAACTTCAAGAGAGTTTCAGAGGAATCATCGA (123) |
| CARBOXYL- | GATAATGGAAATATTGAACTTCAAGAGAGTTCAATATTTCCATTATC (124) |
| TERMINAL | GTTCTTCATTTAAATGATATTCAAGAGATATCATTTAAATGAAGAAC (125) |
| HYDROLASE 16 | GTTAACAAACACATAAAGTTTCAAGAGAACTTTATGTGTTTGTTAAC (126) |
| USP9X | GTTAGAGAAGATTCTTCGTTTCAAGAGAACGAAGAATCTTCTCTAAC (127) |
| | GTTGATTGGACAATTAAACTTCAAGAGAGTTTAATTGTCCAATCAAC (128) |
| | GGTTGATACCGTAAAGCGCTTCAAGAGAGCGCTTTACGGTATCAACC (129) |
| | GCAATGAAACGTCCAATGGTTCAAGAGACCATTGGACGTTTCATTGC (130) |
| USP9Y | AGCTAGAGAAAATTCTTCGTTCAAGAGACGAAGAATTTTCTCTAGCT (131) |
| | GATCCTATATGATGGATGATTCAAGAGATCATCCATCATATAGGATC (132) |
| | GTTCTTCTTGTCAGTGAAATTCAAGAGATTTCACTGACAAGAAGAAC (133) |
| | CTTGAGCTTGAGTGACCACTTCAAGAGAGTGGTCACTCAAGCTCAAG (134) |
| UBIQUITIN | GACCGGCCAGCGAGTCTACTTCAAGAGAGTAGACTCGCTGGCCGGTC (135) |
| CARBOXYL- | GGACCTGGGCTACATCTACTTCAAGAGAGTAGATGTAGCCCAGGTCC (136) |
| TERMINAL | CTCTGTGGTCCAGGTGCTCTTCAAGAGAGAGCACCTGGACCACAGAG (137) |
| HYDROLASE 5 | GACCACACGATTTGCCTCATTCAAGAGATGAGGCAAATCGTGTGGTC (138) |
| UBIQUITIN | TGGCTTGTTTATTGAAGGATTCAAGAGATCCTTCAATAAACAAGCCA (139) |
| CARBOXYL- | GTGAATTTGGGGAAGATAATTCAAGAGATTATCTTCCCCAAATTCAC (140) |
| TERMINAL | CGCTATAGCTTGAATGAGTTTCAAGAGAACTCATTCAAGCTATAGCG (141) |
| HYDROLASE 26 | GATATCCTGGCTCCACACATTCAAGAGATGTGTGGAGCCAGGATATC (142) |
| KIAA1097 | GAGCCAGTCGGATGTAGATTTCAAGAGAATCTACATCCGACTGGCTC (143) |
| | GTAAATTCTGAAGGCGAATTTCAAGAGAATTCGCCTTCAGAATTTAC (144) |
| | GCCCTCCTAAATCAGGCAATTCAAGAGATTGCCTGATTTAGGAGGGC (145) |
| | GTTGAGAAATGGAGTGAAGTTCAAGAGACTTCACTCCATTTCTCAAC (146) |
| UBIQUITIN | GCTTGGAAAATGCAAGGCGTTCAAGAGACGCCTTGCATTTTCCAAGC (147) |
| SPECIFIC | CTGCATCATAGACCAGATCTTCAAGAGAGATCTGGTCTATGATGCAG (148) |
| PROTEASE 22 | GATCACCACGTATGTGTCCTTCAAGAGAGGACACATACGTGGTGATC (149) |
| (USP22) | TGACAACAAGTATTCCCTGTTCAAGAGACAGGGAATACTTGTTGTCA (150) |
| UBIQUITIN- | GAAATATAAGACAGATTCCTTCAAGAGAGGAATCTGTCTTATATTTC (151) |
| SPECIFIC | CCCATCAAGTTTAGAGGATTTCAAGAGAATCCTCTAAACTTGATGGG (152) |
| PROCESSING | GGTGTCCCATGGGAATATATTCAAGAGATATATTCCCATGGGACACC (153) |
| PROTEASE 29 | GAATGCCGACCTACAAAGATTCAAGAGATCTTTGTAGGTCGGCATTC (154) |
| CYLD | CAGTTATATTCTGTGATGTTTCAAGAGAACATCACAGAATATAACTG (155) |
| | GAGGTGTTGGGGACAAAGGTTCAAGAGACCTTTGTCCCCAACACCTC (156) |
| | GTGGGCTCATTGGCTGAAGTTCAAGAGACTTCAGCCAATGAGCCCAC (157) |
| | GAGCTACTGAGGACAGAAATTCAAGAGATTTCTGTCCTCAGTAGCTC (158) |
| UBIQUITIN | TCAGCAGGATGCTCAGGAGTTCAAGAGACTCCTGAGCATCCTGCTGA (159) |
| CARBOXYL- | GAAGTTCTCCATCCAGAGGTTCAAGAGACCTCTGGATGGAGAACTTC (160) |
| TERMINAL | GCCGGTCCCCACCAGCAGCTTCAAGAGAGCTGCTGGTGGGGACCGGC (161) |
| HYDROLASE 2 | CACTCGGGAGTTGAGAGATTTCAAGAGAATCTCTCAACTCCCGAGTG (162) |
| UBIQUITIN | GCCCTTGGGTCTGTTTGACTTCAAGAGAGTCAAACAGACCCAAGGGC (163) |
| SPECIFIC | CTCAACACTAAACAGCAAGTTCAAGAGACTTGCTGTTTAGTGTTGAG (164) |
| PROTEASE 3 | GATTTCATTGGACAGCATATTCAAGAGATATGCTGTCCAATGAAATC (165) |
| (USP3) | CATGGGGCACCAACTAATTTTCAAGAGAAATTAGTTGGTGCCCCATG (166) |
| UBIQUITIN | GGTGTCTCTGCGGGATTGTTTCAAGAGAACAATCCCGCAGAGACACC (167) |
| CARBOXYL- | AGTTCAGTAGGTGTAGACTTTCAAGAGAAGTCTACACCTACTGAACT (168) |
| TERMINAL | GAGTTCCTGAAGCTCCTCATTCAAGAGATGAGGAGCTTCAGGAACTC (169) |
| HYDROLASE 23 | GGATTTGCTGGGGGCAAGGTTCAAGAGACCTTGCCCCCAGCAAATCC (170) |
| UBP-32.7 | CTCAGAAAGCCAACATTCATTCAAGAGATGAATGTTGGCTTTCTGAG (171) |
| | CGCATTGTAATAAGAAGGTTTCAAGAGAACCTTCTTATTACAATGCG (172) |
| | GGGAGGAAAATGCAGAAATTTCAAGAGAATTTCTGCATTTTCCTCCC (173) |
| | TTACAAATTTAGGAAATACTTCAAGAGAGTATTTCCTAAATTTGTAA (174) |
| HOMO SAPIENS | GTTATGAATTGATATGCAGTTCAAGAGACTGCATATCAATTCATAAC (175) |
| UBIQUITIN | GTGATAACACAACTAATGGTTCAAGAGACCATTAGTTGTGTTATCAC (176) |
| SPECIFIC | GTAGAGGAGAGTTCTGAAATTCAAGAGATTTCAGAACTCTCCTCTAC (177) |
| PROTEASE 13 | GCCTCTAATCCTGATAAGGTTCAAGAGACCTTATCAGGATTAGAGGC (178) |
| (ISOPEPTIDASE | |
| T-3) | |
| UBIQUITIN | GATGATCTTCAGGCTGCCATTCAAGAGATGGCAGCCTGAAGATCATC (179) |
| CARBOXYL- | GTATGGACAAGAGCGTTGGTTCAAGAGACCAACGCTCTTGTCCATAC (180) |
| TERMINAL | CGAACCCTTCTGGAACAGTTTCAAGAGAACTGTTCCAGAAGGGTTCG (181) |
| HYDROLASE 28 | GTGGCATGAAGATTATAGTTTCAAGAGAACTATAATCTTCATGCCAC (182) |
| UBIQUITIN | GGTGAACAAGGACAGTATCTTCAAGAGAGATACTGTCCTTGTTCACC (183) |
| CARBOXYL- | GCAATAGAGGATGATTCTGTTCAAGAGACAGAATCATCCTCTATTGC (184) |
| TERMINAL | TCTGTGAATGCCAAAGTTCTTCAAGAGAGAACTTTGGCATTCACAGA (185) |
| HYDROLASE 14 | CACACCAGGGAAGGTCTAGTTCAAGAGACTAGACCTTCCCTGGTGTG (186) |
| DUB1 | GCAGGAAGATGCCCATGAATTCAAGAGATTCATGGGCATCTTCCTGC (187) |
| | GAATGTGCAATATCCTGAGTTCAAGAGACTCAGGATATTGCACATTC (188) |
| | TGGATGATGCCAAGGTCACTTCAAGAGAGTGACCTTGGCATCATCCA (189) |
| | GCTCCGTGCTAAACCTCTCTTCAAGAGAGAGAGGTTTAGCACGGAGC (190) |
| MOUSE USP27 | GCCTCCACCTCAACAGAGGTTCAAGAGACCTCTGTTGAGGTGGAGGC (191) |
| HOMOLOG | CTGCATCATAGACCAAATCTTCAAGAGAGATTTGGTCTATGATGCAG (192) |
| | GATCACTACATACATTTCCTTCAAGAGAGGAAATGTATGTAGTGATC (193) |
| | GTAAAGAGAGCAGAATGAATTCAAGAGATTCATTCTGCTCTCTTTAC (194) |
| UBIQUITIN | CGCGGGGCGCAGTGGTATCTTCAAGAGAGATACCACTGCGCCCCGCG (195) |
| CARBOXYL- | CAGAAGGCAGTGGGGAAGATTCAAGAGATCTTCCCCACTGCCTTCTG (196) |
| TERMINAL | GCCTGGGAGAATCACAGGTTTCAAGAGAACCTGTGATTCTCCCAGGC (197) |
| HYDROLASE 4 | ACCAGACAAGGAAATACCCTTCAAGAGAGGGTATTTCCTTGTCTGGT (198) |
| TRE-2 | CACATCCACCACATCGACCTTCAAGAGAGGTCGATGTGGTGGATGTG (199) |
| | GTCACAACCCAAGACCATGTTCAAGAGACATGGTCTTGGGTTGTGAC (200) |
| | CTCAACAGGACAAATCCCATTCAAGAGATGGGATTTGTCCTGTTGAG (201) |
| | TAGATCAATTATTGTGGATTTCAAGAGAATCCACAATAATTGATCTA (202) |
| UBIQUITIN | GGAACACCTTATTGATGAATTCAAGAGATTCATCAATAAGGTGTTCC (203) |
| CARBOXYL- | CTTTAACAGAAATTGTCTCTTCAAGAGAGAGACAATTTCTGTTAAAG (204) |
| TERMINAL | CCTATGCAGTACAAAGTGGTTCAAGAGACCACTTTGTACTGCATAGG (205) |
| HYDROLASE 15 | GATCTTTTCTTGCTTTGGATTCAAGAGATCCAAAGCAAGAAAAGATC (206) |
| (UNPH-2). | |
| KIAA1372 | CAGCATCCTTCAGGCCTTATTCAAGAGATAAGGCCTGAAGGATGCTG (207) |
| | GATAGTGACTCGGATCTGCTTCAAGAGAGCAGATCCGAGTCACTATC (208) |
| | GACATCACAGCCCGGGAGTTTCAAGAGAACTCCCGGGCTGTGATGTC (209) |
| | GGACACAGCCTATGTGCTGTTCAAGAGACAGCACATAGGCTGTGTCC (210) |
| BRCA1 | GTGGAGGAGATCTACGACCTTCAAGAGAGGTCGTAGATCTCCTCCAC (211) |
| ASSOCIATED | CTCTTGTGCAACTCATGCCTTCAAGAGAGGCATGAGTTGCACAAGAG (212) |
| PROTEIN-1 | ACAGGGCCCCTGCAGCCTCTTCAAGAGAGAGGCTGCAGGGGCCCTGT (213) |
| | GAAGACCTGGCGGCAGGTGTTCAAGAGACACCTGCCGCCAGGTCTTC (214) |

Suitable siRNA sequences for the knockdown of a given target gene are well known in the art (e.g. the particular siRNA sequences mentioned in Lee N. S. et al., J. Nat. Biotechnol. 20(5):500-5 (2002) gcctgtgcctcttcagctacc (SEQ ID NO:215) and gcggagacagcgacgaagagc (SEQ ID NO:216) and in Du, Q. et al., Nucl. Acids Res. 21; (2005) cttattggagagagcacga (SEQ ID NO:217)) or can readily be determined by the skilled artisan.

A preferred embodiment of the method (1) or (2) of the invention concerns the following steps:
1. Generation of the short hairpin DNA containing the antisense- and sense-strand of the coding region of a gene (e.g. firefly luciferase; p53). Antisense and sense-strand are separated by a spacer of 5 to 9 bp.
2. Generation of constructs for the expression of the above mentioned shRNA under the control of a constitutive or inducible promoter (Pol II or Pol III dependent).
3. Insertion of the mentioned expression constructs into an exchange vector and subsequent insertion of the exchange vector into a ubiquitously expressed locus in ES cells by RMCE.
4. Analysis of the constitutive and inducible inhibition of gene expression (e.g. firefly luciferase; p53) in ES cells (e.g. through Western blot analysis).
5. Generation of mice using the mentioned ES cells and analysis of the inhibition of gene expression in several tissues (e.g. firefly luciferase; p53; e.g. through Western blot analysis).

The vector according to embodiment (4) of the invention is suitable for stable or transient integration. Said vector is suitable for gene transfer.

The technology of the present application provides for the following advantages:

(i) A stable and body wide inhibition of gene expression by generating transgenic animals (such as mice).
(ii) A reversible inhibition of gene expression using the inducible constructs.

We showed that high efficient (>90%) RMCE at the ubiquitously expressed Rosa26 locus. The following features where combined in the RMCE strategy of the invention:
1. We utilized Flp mediated RMCE using a wild type Flp target site (FRT) in combination with an inverted F3 site. The F3 sequence was generated by systematic mutagenesis of the 8 bp spacer localized between the Flp binding elements (Schlake & Bode (1994) Biochemistry 33, 12746-12751.). The F3/F3 couple is recombined by FLP with the same efficiency as two wild type recombinase recognition sites (RRS) whereas recombination of a FRT/F3 pair is not catalyzed (Seibler & Bode (1997) Biochemistry 36, 1740-1747.). This characteristic contrasts other pairs of wild type and mutant RRS such as loxp/lox511 that exhibit a residual recombination capacity (Lauth et al., 2002, Nucleic Acids Res. 30:e115).
2. We included a constitutive FLPe expression cassette on the targeting vector to provide sufficient recombinase activity until successful RMCE of the exchange vector. Thus, incomplete recombination intermediates should be avoided.
3. The positive selection marker along with a splice acceptor site on the exchange vector lacks a functional promoter. Thus, expression of the selection marker should only be mediated by the endogenous Rosa26 promoter following successful RMCE, but not through random integration of the exchange vector.
4. The fluorescent protein expression cassette on the targeting vector should allow for the detection of RMCE in early embryos, avoiding long term culture in medium containing antibiotics.

The methods (1) to (3) may further (besides step (a) and (b) defined above) comprise one or more of the steps (c) isolating the eukaryotic cells, preferably the ES cells having the desired fuctional exchange cassette integrated into the Rosa26 locus; and/or (d) modifying the integrated precursor of the exchange cassette and isolating (ES) cells having the desired modified functional exchange cassette.

The steps (a) and (b) of the methods (1) to (3) are preferably performed in vitro. The step (c) may be performed in vitro and in vivo.

The invention also provides a method for preparing a transgenenic multi-cell organism having a modified Rosa26 locus which comprises utilizing the method as defined in (1) to (3) above. This includes a method for preparing a non-human mammal comprising modifying starting ES cells according to steps (a) to (c). The ES cells may subsequently processed according one or more of the following steps:

(d) the ES cells obtained in steps (b) or (c) are injected into blastocysts; and/or
(e) transgenic non-human animals carrying one or more functional genes of interest at the Rosa26 locus are generated (viz. by well known breeding procedures).

The transgenic multi-cell organisms and non-human mammals obtainable by the method (6) and (7), respectively; preferably have an operatively functional gene expression cassette (as defined above) integrated into its Rosa26 locus. Such transgenic multi-cell organisms and non-human mammals are suitable for gene function studies, drug development, as disease model animals, etc.

The invention is further explained by the following examples and the attached figures, which are, however not to be construed so as to limit the invention.

### EXAMPLES

### Materials and Methods

Cell culture: Culture and targeted mutagenesis of ES cells were carried out as described in Hogan et al., (Cold Spring Harbor Laboratory Press, Cold Spring Harbor N.Y.), pp. 253-289 with ES cell lines derived from both inbred and F1 embryos (Examples 1 and 2). In Example 3 Art4.12 ES cells (Seibler et al., Nucl. Acid Res., 31(4):e12 (2003) were used.

Mice: All mice were kept in the animal facility at Artemis Pharmaceuticals GmbH in microisolator cages (Tecniplast Sealsave). B6D2F1 Mice for the generation of tetraploid blastocysts were obtained from Janvier. The polb^{flox}/rosa(CreER^{T2}) and ect2^{flox}/rosa(CreER^{T2}) mice were generated by breeding of rosa(CreER^{T2}) ES mice with βT14 (Gu et al., Science, 265, 103-106.), respectively.

Production of ES mice by tetraploid embryo complementation: The production of mice by tetraploid embryo complementation was essentially performed as described (Eggan et al., Proc Natl Acad Sci USA, 98, 6209-6214.).

Ligand administration: 100 mg Tamoxifen-free base (Sigma, T5648) was suspended in 100 μl Ethanol and solved in 1 ml sunflower oil (Sigma). This 10 mg/100 μl tamoxifen solution was sonicated for 1-2 minutes and then stored at −20° C. For p.o. administration the solution was thawed at 55° C. and administrated to 4-8 week old mice by a feeding needle (FST Fine Science Tools GmbH, 18061-20).

Western blot analysis: Western blot analysis was performed using SDS-PAGE (NuPAGE, Invitrogen) and the Breeze Immunodetection System (Invitrogen) according to the manufacturer protocols. Immunodetection was done using sc-543 (HC-20, Santa Cruz Biotechnology, Inc.) against ER, PRB-106C against cre, actin sc-1616 Actin (1-19) against actin and rabbit polyclonal IgG (Santa Cruz Biotechnology, Inc.) antibodies.

X-Gal staining on tissue sections: To detect beta-galactosidase activity, tissues were embedded in Tissue Tec OCT (Sakura Finetek Europe B.V., The Netherlands), frozen on dry ice and cut into microsections. The sections were mounted onto slides and dried for 1-4 hours at room temperature. Sections were fixed for 5 min at room temperature in fixing solution (0.2% glutaraldehyde, 5 mM EGTA, 2 mM MgCl₂ in 0.1 M PB ((0.1 M K₂HPO₄, pH 7.3)) and washed three times for 15 min at room temperature in washing buffer (2 mM MgCl₂, 0.02% Nonidet-40 in 0.1 M PB). Subsequently, tissues were stained for beta-galactosidase activity over night at 37° C. using X-Gal solution (0.6 mg/ml X-Gal (predissolved in DMSO), 5 mM potassium hexacyanoferrat III, 5 mM potassium hexacyanoferrat II, in washing buffer). Sections were washed twice for 5 min at room temperature in PBS, counterstained with Nuclear Fast Red Solution for 10 min, rinsed shortly in aqua dest., dehydrated through a graded ethanol series and mounted in Eukitt (Sigma, Germany).

### Example 1

### Reference Example

CreER Rosa-targeting vector: A 129 SV/EV-BAC library (Incyte Genomics) was screened with a probe against exon2 of the Rosa26 locus (amplified from mouse genomic DNA using Rscreen1s (GACAGGACAGTGCTTGTTTAAGG) (SEQ ID NO:1) and Rscreen1as (TGACTACACAATATTGCTCGCAC) (SEQ ID NO:2)). Out of the identified BACclone a 11 kb EcoRV subfragment was inserted into the HindIII site of pBS. Two fragments (a 1 kb SacII/XbaI- and a 4 kb XbaI-fragment, SEQ ID Nos:4 and 5, respectively) were used as homology arms and inserted into a vector containing a FRT-flanked neomycin resistance gene (unpublished) to generate the basic Rosa26 targeting vector. The CAGGS-promoter (SEQ ID NO:6, nucleotides 1-1616) or a splice acceptor site (SA) from adenovirus (Friedrich G., Soriano P., Genes Dev., 5:1513-23 (1991)) were inserted between the 5′ arm and the FRT flanked neomycin resistance gene. The CreER^{T2} and a polyadenylation site (pA; SEQ ID NO:6, nucleotides 3921-4099) were cloned 3′ of the SA or the CAGGS-promoter. The vector is free of a transcriptional stop sequence 5′ to the CAGGS-promoter

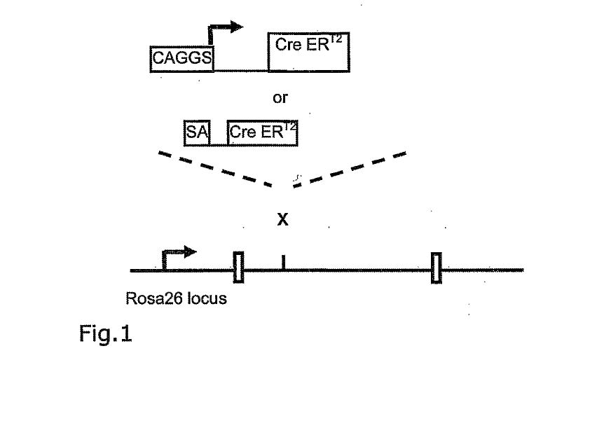

A CreER^{T2} gene (Feil et al., (1997) Biochem Biophys Res Commun., 237, 752-757) under the control of the CAGGS-promoter (Okabe, Fabs Letters 407:313-19 (1997)) was inserted into the rosa26 locus by homologous recombination in ES cells by utilizing the CreER Rosa-targeting vector as described above (FIG. 1). In addition to the CreER<gene a splice acceptor sequence (Friedrich and Soziano (1991), Genes Dev., 9, 1513-1523) was introduced as a control for the endogenous activity of the rosa26 gene promoter (FIG. 1). A loxP-flanked hygromycin resistance gene was introduced into the second allele of rosa26 to provide test substrate for Cre ER72 (Seibler et al., Nucl. Acids. Res. Feb. 15, 2003, 31(4):(12) (2003)), in press). ES cells modified at both rosa26 alleles were injected into tetraploid blastocysts and completely ES cell derived mice were generated (Eggan et al., (2001). PNAS, 98, 6209-6214). Rosa(SA-CreER^{T2}/reporter) and Rosa(CAGGS-CreER^{T2}/reporter) mice were fed with daily 5 mg Tamoxifen for 5 days and recombination of the reporter was analyzed 3 days after the last administration. Southern analysis of genomic DNA from different organs showed up to 50% recombination in the Rosa(SA-CreER^{T2}/reporter) mice and up to 90% recombination in the rosa(CAGGS-CreER^{T2}/reporter) mice, respectively (FIG. 2A). As the second substrate, we used the loxP flanked DNA polymerase β gene segment (polβ^{flox}) (Gu et al., (1994). Science, 265, 103-106). The polβ^{flox}/rosa(SA-CreER^{T2}) and polβ^{flox}/rosa(CAGGS-CreER^{T2} mice were fed with 5 mg tamoxifen per day for 5 days and analyzed 3 days later. Southern blot analysis revealed that the loxP-flanked polymerase 8 gene segment was excised in more than 90% of cells in all organs except brain in the rosa(SA-CreER^{T2}/reporter) mice (FIG. 2B). In contrast, the degree of inducible recombination was significantly higher in rosa(CAGGS-CreER^{T2}/reporter) mice, reaching 100% efficiency in most organs and up to 70% in brain.

To investigate the pattern and level of CreER^{T2} expression in rosa(SA-CreER^{T2}) and rosa(CAGGS-CreER^{T2}) mice, we performed Western analysis using antibodies specific for Cre. The 74 kDa band corresponding to the CreER^{T2} fusion protein was detectable in all organs of rosa(CAGGS-CreER^{T2}) mice, including brain (FIG. 3). In contrast, the CreER^{T2} expression level in rosa(SA-CreER^{T2}) mice was significantly lower compared to the rosa(CAGGS-CreER^{T2}) strain and appeared to be undetectable in brain (FIG. 3).

### Example 2

### Reference Example

FABP-Cre Rosa-targeting vector (SEQ ID NO:8): The splice acceptor site from adenovirus (SEQ ID NO:8, nucleotides 18569-18689) was inserted into the basic Rosa26 targeting vector described in 1. above. Into the SwaI and AscI restriction sites of the resulting plasmid was inserted a 3195 bp Xba_{blunt}/AscI DNA fragment comprising in 5′ to 3′ order the polyadenylation signal from the human growth hormone gene (SEQ ID NO:8, nucleotides 18760-688; Bond et al, Science 289:1942-1946 (2000)), a modified Fabpl promoter (SEQ ID NO:8, nucleotides 702-1481; Fabpl^{4x at -132}; Simon et al., J. Biol. Chem. 272:10652-10663 (1997)), a synthetic intron (SEQ ID NO:8, nucleotides 1521-1758), the Cre coding sequence (SEQ ID NO:8, nucleotides 1778-2830) and a synthetic polyA signal (SEQ ID NO:8, nucleotides 2888-3066).

A Cre gene under the control of the Fabpl^{4x at -132}-promoter (SEQ ID NO:8; FIG. 4) was inserted into the Rosa26 locus by homologous recombination in F1 ES cells carrying a Cre reporter substrate in the second Rosa26 allele. LacZ expression from the reporter construct (SEQ ID NO:9; FIG. 5) is activated upon Cre-mediated recombination. Targeted ES cells were injected into tetraploid blastocysts to generate FABP-Cre/reporter-substrate double transgenic ES mice. The Cre recombination pattern in these mice was examined by analyzing beta-galactosidase activity in tissues sections (FIG. 6). Cre-mediated recombination in these mice was restricted to the intestinal epithelium, liver and part of the cells in the epithelium of the tubuli in the kidney, thus exactly reflecting the expression pattern of the endogenous Fabpl gene (Simon et al., J. Biol. Chem., 272:10652-10663 (1997)).

### Example 3

### Rosa Targeting and Exchange Vectors

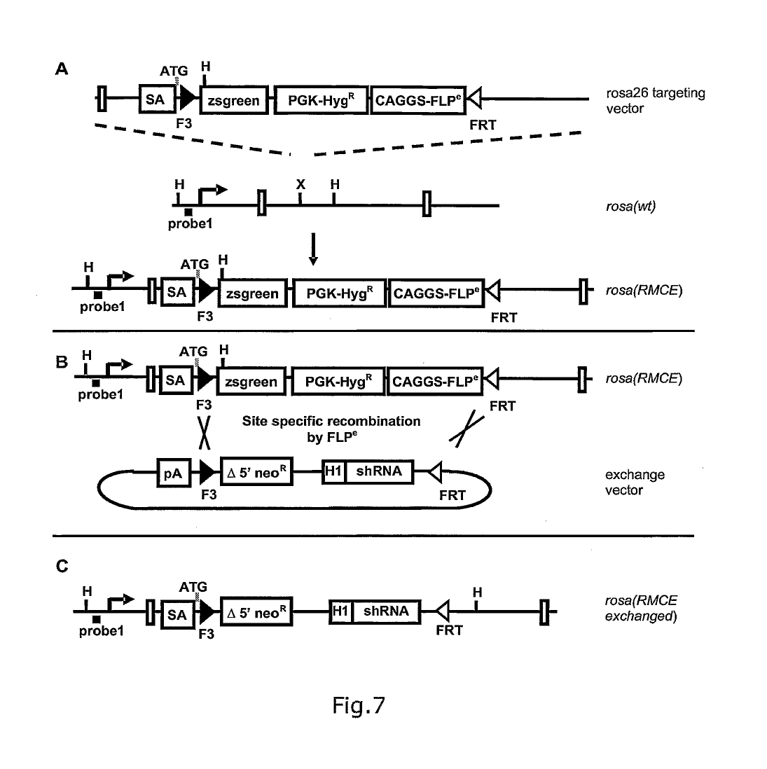

Rosa26 RMCE targeting vector (SEQ ID NO:11): A 129 SV/EV-BAC library (Incyte Genomics) was screened with a probe against exon2 of the Rosa26 locus (SEQ ID NO:3). The exon2 probe was amplified from mouse genomic DNA using primers Rscreen1s (GACAGGACAGTGCTTGTTTAAGG; SEQ ID NO:1) and Rscreen1as (TGACTACACAATATTGCTCGCAC; SEQ ID NO:2). A 11 kb EcoRV fragment isolated from the identified BAC clone a was inserted into the HindIII site of pBS. Two subfragments from the 11 kb EcoRV fragment, the 1 kb SacII/XbaI- (SEQ ID NO:4) and the 4 kb XbaI-fragment (SEQ ID NO:5), were used as homology arms and inserted into a vector containing a FRT-flanked neomycin resistance gene (unpublished) to generate the basic Rosa26 targeting vector (SEQ ID NO:10). A splice acceptor site (SA) from adenovirus (Friedrich G., Soriano P., Genes Dev., 5:1513-23 (1991)) was inserted between the 5′ arm and the FRT flanked neomycin resistance gene. The neomycin was deleted by Flp-mediated deletion in bacteria (Buchholz et al., Nucleic Acids Res. 1996, 24:3118-9). The final Rosa(RMCE) targeting vector (SEQ ID NO:11, FIG. 7A) was generated by standard cloning procedures and has the following order in 5′ to 3′ direction: a ATG start codon, a F3 site (Schlake & Bode (1994) Biochemistry 33, 12746-12751; (SEQ ID NO:11, nucleotides 1292-1339)), a zsgreen ORF (Clontech; SEQ ID NO:11, nucleotides 1407-2099), a synthetic polyA signal (SEQ ID NO:11, nucleotides 2121-2299), a PGK-hygro resistance gene (SEQ ID NO:11, nucleotides 2314-4335), a CAGGS-promoter (SEQ ID NO:11, nucleotides 4397-6012), a Flpe-recombinase gene (Buchholz et al., Nat. Biotechnol. 1998, 16:657-62.), a synthetic polyA signal (SEQ ID NO:11, nucleotides 7728-7906), and a FRT site (SEQ ID NO:11, nucleotides 7922-7969) 5′ of the 3′ homology arm.

Exchange vector (SEQ ID NO:12): The vector contains the F3 site and the FRT site in the same configuration as in the Rosa26 targeting vector described above. The vector was generated using standard cloning procedures and has the following order in 5′ to 3′ direction: a synthetic polyA signal (SEQ ID NO:12, nucleotides 23-201), a F3-site (SEQ ID NO:12, nucleotides 216-263), a neomycin-resistance gene lacking the start ATG (SEQ ID NO:12, nucleotides 271-1559), a H1-promoter (SEQ ID NO:12, nucleotides 1763-1996), a hairpin sequence (SEQ ID NO:12, nucleotides 1997-2051), and a FRT site (SEQ ID NO: 12, nucleotides 2161-2208).

Cell culture: ES cell culture and homologous recombination were carried out as previously described (Hogan et al., (Cold Spring Harbor Laboratory Press, Cold Spring Harbor N.Y.), pp. 253-289.)

Transfection of cells with the exchange vector: 1 day before transfection, 2×10⁵ ES cells were plated on a 3 cm dish in 2 ml medium. Before transfection 2 ml fresh medium was given to the cells. 3 μl Fugene6 Reagent (Roche; Cat No. 1 814 443) was mixed with 100 μl serum free medium (OptiMEM 1 with Glutamax-I Invitrogen; Cat. No. Cat. No. 51985-035) and incubated for 5 min. 100 μl of the Fugene/OptiMEM solution was added to 2 μg circular DNA (c=0.33 μg/μl) and incubated for 15 min. This transfection complex was added drop wise to the medium and mixed by a circuiting movement. Fresh medium was added to the transfected cells the following day. From day 2, the medium was changed daily replaced by medium containing 250 μg/ml G418 (Geneticin; Invitrogen; Cat. No. 10131-019). 7 days after transfection, single clones were isolated by standard procedures as described (Hogan et al., (Cold Spring Harbor Laboratory Press, Cold Spring Harbor N.Y.), pp. 253-289.).

The targeting vector to prepare the Rosa26 locus for RMCE is depicted in FIG. 7A. The vector carries a FLP^{e} expression cassette to provide the recombinase for RMCE. The hygromycine resistance gene was used for positive selection of homologous recombinant clones. In addition, a zsGreen gene was placed between the FRT and F3 sites to allow for the identification of recombinant clones that have not undergone RMCE following secondary transfection of the exchange vector. The splice acceptor site (SA) and the ATG start codon should facilitate expression of the truncated neomycine resistance gene (Δ5′neo^{R}) on the exchange vector by employing the endogenous rosa26 promoter following RMCE.

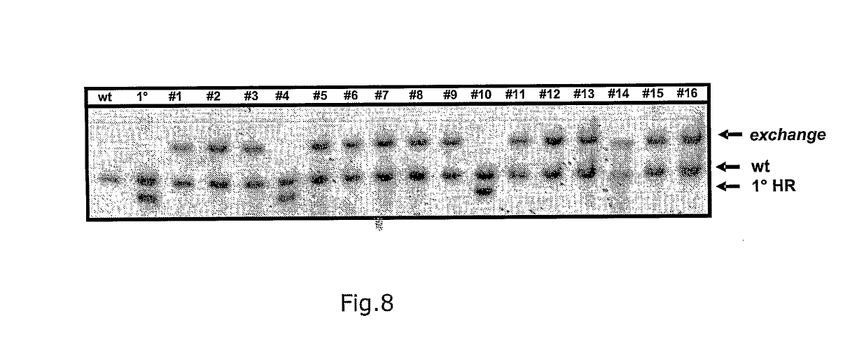

The hybrid ES cell line ART4.12 ([C57BL/6×129S6/SvEvTac] F1) was used for homologous recombination, since these lines are capable to derive completely ES cell derived mice (ES mice) through tetraploid blastocyst complementation with high efficiency (Seibler et al., Nucl. Acid Res., 31(4):e12 (2003). ART4.12 cells where transfected with the rosa26 targeting vector and incubated in cell culture medium containing hygromycin B. Independent recombinant Rosa(RMCE) ES cell clones were obtained at a frequency of 2% as verified by Southern blot analysis (FIG. 8, first and second lane.

The exchange vector (FIG. 7B) carries the FRT and F3 sites together with a truncated neo^{R} gene for positive selection of RMCE. The shRNA expression cassette served as a test transgene for targeted integration into the Rosa26 locus. The upstream polyA signal was included to prevent expression of the truncated neo^{R} gene in ES cells carrying randomly integrated vectors. The configuration of the targeted Rosa26 locus following RMCE is depicted in FIG. 7C.

Rosa(RMCE) ES cells where transfected with the exchange vector and selected in medium containing G418. Southern blot analysis of G418 resistant colonies revealed that successful RMCE had occurred in >90% of clones (FIG. 8). This is the first demonstration of efficient RMCE for targeted transgenesis at a ubiquitously expressed locus.

ShRNA transgenic ES cells were injected into tetraploid blastocysts and ES cell derived mice were obtained three weeks later at a frequency of 3%. Real time PCR analysis of 15 week old mice indicated a >80% reduction of leptin receptor mRNA in most organs, indicating that the shRNA transgene is ubiquitously expressed (Seibler et al. 2005, Nucl Acids Res 33(7):e67).

### Example 4

lacZ specific shRNA (nucleotide 1998-2055, SEQ ID NO:218) under the control of the human U6 promoter through RMCE was introduced into ART4.12/rosa26(RMCE) ES cells (Seibler et al. 2005, Nucl Acids Res 33(7):e67). Southern blot analysis of G418 resistant clones revealed that successful RMCE had occurred in >90% of clones. Recombinant ES cells were injected into tetraploid blastocysts and ES cell derived mice were derived. A highly expressed β-galactosidase gene was provided through breeding using a mouse strain carrying lacZ (nucleotide 2161-5678, SEQ ID NO:219) under the control of the ubiquitous CAGGS promoter, that had been placed into the Rosa26 promoter. X-Gal staining on tissue sections revealed a strong, uniform expression of lacZ under the control of the CAGGS promoter in every single cell, whereas the presence of the shRNA construct resulted in marked reduction of β-galactosidase activity in the vast majority of cells (Seibler et al. 2005, Nucl Acids Res 33(7):e67). These results indicate that the lacZ specific shRNA is sufficiently expressed to mediate efficient RNAi in all organs.

### Example 5

A CAGGS-Fluc (nucleotide 2100-5983, SEQ ID NO:218) expression cassette was inserted into the ES cell genome using RMCE at the rosa26 locus (Seibler et al. 2005, Nucl Acids Res 33(7):e67). Again, successful RMCE had occurred in >90% of clones as confirmed by Southern blot analysis. Recombinant ES cells were injected into blastocysts and mice were obtained upon transfer of blastocysts into pseudopregnant females. The Fluc-specific shRNA gene under the control of the human U6 promoter and the CAGGS-Fluc transgene were combined through breeding of mice. Measurement of luciferase activity in protein extracts from various organs revealed a strong reduction of luciferase activity in the presence, but not in the absence of the shRNA, indicating that both transgenes are ubiquitously expressed (Seibler et al. 2005, Nucl Acids Res 33(7):e67).

1-20. (canceled)

21. Method for generating transgenic eukaryotic cells having an ubiquitous locus modified by an expression cassette comprising a short hairpin RNA construct operatively linked to a promoter or an inactive precursor thereof, which method comprises introducing the expression cassette into the ubiquitous locus of eukaryotic cells by recombinase mediated cassette exchange (RMCE).

22. The method of claim 21, which comprises
(a) introducing a functional DNA sequence into the Rosa26 locus of starting eukaryotic cells by homologous recombination with a targeting vector comprising flanking DNA sequences homologous to the ubiquitous locus and an acceptor DNA, which integrates into the genome of the starting cell, the acceptor DNA comprising two mutually incompatible first recombinase recognition sites (RRSs), and
(b) effecting RMCE of the recombination product of step (a) having RMCE target sites with an exchange vector comprising a donor DNA, which comprises the expression cassette flanked by the same two mutually incompatible first RRSs contained in the acceptor DNA, by utilizing a recombinase which catalyzes recombination between the RRSs of the acceptor and donor DNA.

23. The method of claim 21, wherein the eukaryotic cells are derived from multi-cell organisms selected from the groups of organisms consisting of vertebrates, invertebrates and plants.

24. The method of claim 23, where the eukaryotic cells are vertebrate cells.

25. The method of claim 24, where the vertebrate cells are derived from mammals.

26. The method of claim 25, wherein the mammals are non-human mammals.

27. The method of claim 26, wherein the non-human mammals are rodents.

28. The method of claim 27, wherein the rodents are selected from the group consisting of mouse and rat.

29. The method of claim 24, wherein the vertebrates cells are derived from fish.

30. The method of claim 29, wherein the fish is zebrafish.

31. The method of claim 21, wherein the eukaryotic cells are selected from the group consisting of primary cells and immortalized cells.

32. The method of claim 31, wherein the cells are mammalian embryonic stem (ES cells).

33. The method of claim 21, wherein the ubiquitous locus is selected from the group consisting of Rosa26, Collagen, β-Actin, HPRT, U6, H1, tRNA, and 7SL RNA.

34. The method of claim 33, wherein the ubiquitous locus is a Rosa26 locus.

35. The method of claim 21, which is performed in vitro.

36. The method of claim 21, wherein the promoter is a heterologous promoter.

37. The method of claim 36, wherein the promoter is selected from the group consisting of ubiquitous and tissue specific promoters, either constitutive or inducible.

38. The method of claim 22, wherein the targeting vector, the exchange vector and the expression cassette, independently from each other, further comprises one or more additional functional sequences selected from the group consisting of marker genes, second recombinase recognition sites differing from the first recombinase recognition sites, poly A signal and introns.

39. The method of claim 22, wherein the targeting vector and the exchange vector, independently from each other, further comprises one or more additional functional sequences selected from the group consisting of tags for protein detection, enhancers and selection markers.

40. The method of claim 22, wherein the targeting vector further comprises a gene coding for the recombinase which catalyses recombination between acceptor and donor DNA.

41. The method of claim 22, wherein in step (a) the DNA sequences homologous to the ubiquitous locus are 0.2 to 20 kB long.

42. The method of claim 41, where the DNA sequences are 1 to 10 kB long.

43. The method of claim 22, wherein the mutually incompatible RRS are selected from the group consisting of pairs of mutually incompatible loxP, FRT, and Att sites or variants thereof.

44. The method of claim 43, wherein the mutually incompatible RRS are selected from the group consisting of the following group of mutually incompatible RRS pairs: F3/FRT, F5/FRT, F5/F3, lox/lox511, lox/lox2722, lox66/lox71 and AttB/AttP.

45. The method of claim 22, wherein the recombinase, which may be added to the cell or may be expressed by the cell, is selected from recombinases suitable for cassette exchange of the first RSSs present in the acceptor/donor DNA.

46. The method of claim 45, wherein the recombinase is selected from the group consisting of Cre, Flp, Φ31 and mutants thereof.

47. The method of claim 21, wherein the short hairpin RNA construct comprises one or more additional functional sequences selected from the group consisting of stop and polyadenylation sequences.

48. The method according to claim 37, wherein the ubiquitous promoter is selected from the group consisting of polymerase I, II and III dependent promoters.

49. The method of claim 48, wherein the ubiquitous promoter is selected from the group consisting of polymerase II and III dependent promoters.

50. The method of claim 49, wherein the promoter is selected from the group consisting of a CMV promoter, a CAGGS promoter, a Mx promoter, a PGK promoter, a snRNA promoter, a RNAse P RNA promoter, a tRNA promoter, a 7SL RNA promoter, and a 5 S rRNA promoter.

51. The method of claim 37, wherein the tissue specific promoter is selected from the group consisting of FABP, Lck, CamKII, CD19, Keratin, Albumin, a P2, Insulin, MCK, MyHC, WAP and Col2A promoters.

52. The method of claim 37, wherein the ubiquitous promoter is selected from the group consisting of constitutive and inducible promoters.

53. The method of claim 52, wherein the ubiquitous promoter is a promoter containing an operator sequence selected from the group consisting of tet, Gal4, lac, and RRSs for recombinase mediated control.

54. The method of claim 37, wherein the promoter of the expression cassette is a Pol III dependent promoter.

55. The method of claim 54, wherein the expression cassette is a constitutive H1 or U6, driven shRNA construct suitable to be integrated into a ubiquitously active Pol II dependent locus.

56. The method of claim 55, where the expression cassette is an inducible U6 or H1 driven shRNA construct suitable to be integrated into a ubiquitously active Pol II dependent locus.

57. The method of claim 37, wherein the promoter of the expression cassette is a Pol II dependent promoter.

58. The method of claim 57, wherein the expression cassette is an inducible CMV driven shRNA construct suitable to be integrated into a ubiquitously active Pol II dependent locus.

59. The method of claim 21, wherein the short hairpin RNA construct or the inactive precursor thereof comprises at least one segment corresponding to a short hairpin RNA (ShRNA).

60. The method of claim 59, wherein the short hairpin RNA construct comprises at least one shRNA segment having a DNA sequence selected from the group consisting of A-B-C and C-B-A, wherein
A is a 15 to 35 bp DNA sequence with at least 95% complementarily to the gene to be knocked own;
B is a spacer DNA sequence having 5 to 9 bp forming the lop of the expressed RNA hair pin molecule, and
C is a 15 to 35 bp DNA sequence with at least 85% complementarily to the sequence A.

61. The method of claim 21, wherein the short hairpin RNA construct or the inactive precursor thereof comprises at least two complementary short interfering RNA (siRNA) strands.

62. The method of claim 61, wherein the short hair RNA construct comprises at least two siRNA segments selected from the group consisting of A and C, and C and A, each of said at least two siRNA segments being under the control of a separate precursor, wherein
A is a 15 to 35 bp DNA sequence with at least 95% complementarily to the gene to be knocked down;
B is a spacer DNA sequence having a 5 to 9 bp forming the loop of the expressed RNA hair pin molecule, and
C is a 15 to 35 bp DNA sequence with at least 85% complementarily to the sequence A.

63. The method of claim 23, wherein the transgenic eukaryotic cells are derived from mouse and the ubiquitous locus is a Rosa 26 locus.

64. The method of claim 63, wherein the DNA sequences homologous to the Rosa26 locus are derived from the 5′ and 3′ flanking arm of the mouse Rosa26 locus.

65. The method of claim 64, wherein said homologous DNA sequences have the sequences shown in SEQ ID NO:4 and 5.

66. The method of claim 44, wherein the RRSs of the targeting and exchange vectors are F3/Frt and the targeting vector encodes the recombinase Flp or a mutant thereof.

67. The method of claim 66, wherein the recombinase is Flpe.

68. The method of claim 39, where the targeting vector comprises a negative selection marker.

69. The method of claim 39, where the exchange vector comprises a promoter-less positive selection marker.

70. The method of claim 36, where the promoter of the expression cassette is a H1 or H6 promoter.

71. The method of claim 21, where the targeting vector has the sequence shown in SEQ ID NO:11 and the exchange vector has the sequence shown in SEQ ID NO:12 or a variant thereof with modification in the short hairpin RNA construct.

72. The method according to claim 22, which further comprises one or more of the steps
(c) isolating the eukaryotic cells, the ES cells having the desired functional exchange cassette or the inactive precursor integrated into the ubiquitous locus; and
(d) optionally modifying the integrated precursor of the expression cassette to activate the precursor and isolating ES cells having the desired modified functional exchange cassette.

73. An exchange vector comprising the expression cassette and a donor DNA, as defined in claim 22.

74. A eukaryotic cell having a modified ubiquitous locus obtainable by the method of claim 21.

75. A method for preparing transgenenic multi-cell organism having a modified ubiquitous locus which comprises transfecting eukaryotic cells according to the method defined in claim 21.

76. A method for preparing transgenenic multi-cell organism having a modified unbiquitous locus which comprises injecting an exchange vector of claim 73 into an early stage embryo of a non-human mammal having corresponding RMCE target sites.

77. The method of claim 75, wherein the transgenenic multi-cell organism is a non-human mammal, said ubiquitous locus is a Rosa26 locus, and said method comprises modifying an ES cell.

78. The method of claim 75, which further comprises one or more of the steps (e) injecting ES cells obtained in steps (c) or (d) into blastocysts; and (f) generating transgenic non-human multi-cell organisms or non-human mammals carrying one or more functional genes of interest at the Rosa26 locus.

79. A transgenic multi-cell organism or a tissue culture derived therefrom or a transgenic non-human mammal or a tissue culture derived therefrom, which are obtainable by the method of claim 75, respectively, and having an operatively functional gene expression cassette integrated into at least one of its loci.

80. A method for constitutive and/or inducible gene knock down in a multi-cell organism, or in a tissue culture or cells of a cell culture derived from said multi-cell organism, which comprises stably integrating an expression vector as defined in claim 22 into the genome of the living organism, of the tissue culture or of the cells of the cell culture.

81. The method of claim 79, wherein (i) the expression vector is integrated at the Rosa26 locus of the multi-cell organism, tissue culture or cell culture.

82. The method of claim 79, wherein the method for constitutive and/or inducible gene knock down in a vertebrate comprises integrating the expression vector into ES cells of the vertebrate.

83. Method of using the eukaryotic cell of claim 74 for gene function studies and drug development.

84. Method of using the transgenic multi-cell organism, tissue culture or non-human mammal of claim 79 for gene function studies, drug development and disease model animals.
